(19) United States
(12) Patent Application Publication
(10) Pub. No.: US 2008/0206355 A1
(43) Pub. Date: Aug. 28, 2008

(54) Composition comprising a particulate zinc material, a pyrithione or a polyvalent metal salt of a pyrithione and a synthetic cationic polymer

(75) Inventors: James Robert Schwartz, West Chester, OH (US); Eric Scott Johnson, Hamilton, OH (US); Bonnie Theresa King, Alexandria, KY (US); Carl Hinz Margraf, Frankfurt Am Main (DE); Gregory V. Tomos, Loveland, OH (US); David Thomas Warnke, Cincinnati, OH (US); Deborah W. Chang, Mason, OH (US); David Scott Dunlop, Mason, OH (US); Kevin M. Labitzke, Fairfield, OH (US); Sandra Lou Murawski, Fairfield, OH (US); William Jeffrey Gore, Montgomery, OH (US); Theodore Jay Verbrugge, Reily, OH (US); Mark Anthony Brown, Union, KY (US); Timothy Woodrow Coffindaffer, Maineville, OH (US); Afua Asiedua Asante, Holly Springs, NC (US); Robert Lee Wells, Cincinnati, OH (US); Teresa Cuasay Manuel, West Chester, OH (US); Nicholas William Geary, Hamilton, NY (US); Martin Asare, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/890,684

(22) Filed: Aug. 7, 2007

Related U.S. Application Data
(63) Continuation-in-part of application No. 11/216,520, filed on Aug. 31, 2005.
Application No. 11/216,520 is a continuation-in-part of application No. 11/100,648, filed on Apr. 7, 2005.
Application No. 11/100,648 is a continuation-in-part of application No. 10/802,166, filed on Mar. 17, 2004.
Application No. 10/802,166 is a continuation-in-part of application No. 10/454,234, filed on Jun. 4, 2003, now abandoned.
(60) Provisional application No. 60/455,963, filed on Mar. 18, 2003.
(60) Provisional application No. 60/385,794, filed on Jun. 4, 2002.

(51) Int. Cl. A61K 33/42; A61K 33/30; A61K 33/34; A61P 17/00

(52) U.S. Cl. 424/604; 424/642; 424/633

(57) ABSTRACT

The present invention relates to a composition comprising a composition comprising an effective amount of a particulate zinc material; an effective amount of a surfactant including a surfactant with an anionic functional group; an effective amount of a pyrithione or a polyvalent metal salt of a pyrithione; from about 0.025% to about 5% by weight of a water soluble or dispersible, cationic, non-crosslinked, conditioning homopolymer having a cationic charge density of from about 2 meq/gm to about 10 meq/gm; and from about 20% to about 95% of an aqueous carrier, by weight of said composition

### CROSS REFERENCE TO RELATED APPLICATION

This application is a continuation-in-part of U.S. application Ser. No. 11/216,520, filed on Aug. 31, 2005, which is a continuation-in-part of U.S. application Ser. No. 11/100,648, filed on Apr. 7, 2005, which is a continuation-in-part of U.S. application Ser. No. 10/802,166, filed on Mar. 18, 2004, and claims the benefit of U.S. Provisional application Ser. No. 60/455,963, filed on Mar. 17, 2003. This application further claims the benefit of and is a continuation in-part of U.S. application Ser. No. 10/454,234, filed on Jun. 4, 2003 and claims the benefit of U.S. Provisional Application Ser. No. 60/385,794 filed on Jun. 4, 2002.

### FIELD

The present invention relates to a composition comprising an effective amount of a particulate zinc material, a surfactant with an anionic functional group, an effective amount of a pyrithione or a polyvalent metal salt of a pyrithione and a water soluble or dispersible cationic, non-crosslinked, conditioning homopolymer. More particularly, the present invention relates to personal care compositions and methods of treating microbial and fungal infections on the skin or scalp. Even more particularly, the present invention relates to methods for the treatment of dandruff and compositions, which provide improved anti-dandruff activity and improved conditioning.

### BACKGROUND

Of the trace metals, zinc is the second most abundant metal in the human body, catalyzing nearly every bio-process directly or indirectly through inclusion in many different metalloenzymes. The critical role zinc plays can be discerned from the symptoms of dietary deficiency, which include dermatitis, anorexia, alopecia and impaired overall growth. Zinc appears especially important to skin health and has been used (typically in the form of zinc oxide or calamine) for over 3000 years to control a variety of skin problems. Recent data more specifically points to the healing and repairing properties of topical zinc treatment to damaged skin, often resulting in increased rates of healing. There is a growing body of biochemical support for this phenomenon. Since dandruff has been previously shown to represent significant damage to scalp skin, topical zinc treatment could aid in the repair process.

Inorganic salts, such as zinc hydroxycarbonate and zinc oxide, have been employed as bacteriostatic and/or fungistatic compounds in a large variety of products including paints, coatings and antiseptics. However, zinc salts do not possess as high of a level of biocidal efficacy as might be desired for many anti-dandruff and skin care applications.

Despite the options available, consumers still desire a shampoo that provides superior anti-dandruff efficacy versus currently marketed products; as such consumers have found that dandruff is still prevalent. Such a superior efficacy can be difficult to achieve. In addition, consumers also desire a shampoo that provides superior anti-dandruff efficacy along with improved conditioning benefit for dry hair, while not interfering with the cleansing or anti-dandruff efficacy, nor providing negative feel to the hair when it is dried.

Conditioning shampoos comprising various combinations of detersive surfactant and hair conditioning agents are known. These shampoo products typically comprise an anionic detersive surfactant in combination with a conditioning agent such as silicone, hydrocarbon oil, fatty esters, or combinations thereof. These shampoos have become more popular among consumers as a means of conveniently obtaining hair conditioning and hair cleansing performance all from a single hair care product.

Many conditioning shampoos, however, do not provide sufficient deposition of conditioning agents onto hair during the shampooing process. Without such deposition, large proportions of conditioning agent are rinsed away during the shampooing process and therefore provide little or no conditioning benefit. Without sufficient deposition of the conditioning agent on the hair, relatively high levels of conditioning agents may be needed in the shampoo composition to provide adequate hair conditioning performance. Such high levels of a conditioning agent, however, can increase raw material costs, reduce lathering, and present product stability concerns.

Obtaining good deposition of a conditioning agent onto hair is further complicated by the action of detersive surfactants in the shampoo. Detersive surfactants are designed to carry away or remove, oil, grease, dirt, and particulate matter from the hair and scalp. In doing so, the detersive surfactants can also interfere with deposition of the conditioning agent, and carry away both deposited and non deposited conditioning agent during rinsing. This further reduces deposition of the conditioning agent onto the hair after rinsing, thus further reducing hair conditioning performance.

One known method for improving deposition of a hair conditioning agent onto hair involves the use of certain cationic deposition polymers. These polymers may be synthetic, but are most typically natural cellulosic or guar polymers that have been modified with cationic substituents. The cationic charge density of such polymers, especially when used in a shampoo composition, is minimized so as to avoid incompatibility with anionic materials in the shampoo such as anionic surfactant. As such, most shampoos which contain both an anionic detersive surfactant and a cationic deposition polymer will maintain relatively low cationic charge density values for the deposition polymer in order to maintain physical stability of the shampoo composition.

Based on the foregoing, there is a need for a shampoo which provides superior anti-dandruff efficacy and further provides improved conditioning performance in shampoo compositions. None of the existing art provides all of the advantages and benefits of the present invention.

### SUMMARY

An embodiment of the present invention is directed to a composition comprising an effective amount of a particulate zinc material; an effective amount of a surfactant including a surfactant with an anionic functional group; an effective amount of a pyrithione or a polyvalent metal salt of a pyrithione; from about 0.025% to about 5% by weight of a water soluble or dispersible, cationic, non-crosslinked, conditioning homopolymer having a cationic charge density of from about 2 meq/gm to about 10 meq/gm; and from about 20% to about 95% of an aqueous carrier, by weight of said composition.

A further embodiment of the present invention is directed to a composition an effective amount of a particulate zinc material; an effective amount of a surfactant including a surfactant with an anionic functional group; an effective amount of a pyrithione or a polyvalent metal salt of a pyrithione; from about 0.025% to about 5% by weight of a water soluble or dispersible, cationic, non-crosslinked, conditioning copolymer having a cationic charge density of from about 2 meq/gm to about 10 meq/gm; and from about 20% to about 95% of an aqueous carrier, by weight of said composition.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

It has now surprisingly been found, in accordance with the present invention, that anti-dandruff efficacy can be dramatically increased in topical compositions by the combination of an effective amount of a particulate zinc material with a surfactant with an anionic functional group and such topical compositions can further provide improved conditioning by the use of a water soluble or dispersible, cationic, no crosslinked, deposition polymer.

One embodiment of the present invention concerns the surprising discovery that compositions combining certain water soluble or dispersible, cationic, non crosslinked, deposition polymers in combination with surfactants form microscopically-phase separate lyotropic liquid crystals suspended in an aqueous surfactant phase. In use, the dispersed, concentrated polymer lyotropic liquid crystal phase provides improved hair and skin conditioning.

Moreover, without being limited to a particular theory, it appears that when dispersed conditioning agent particles are added to the matrix, the concentrated polymer lyotropic liquid crystal phase provides an improved mechanism for conditioning agent deposition, yielding conditioning agent deposition that results in more conditioning.

The liquid crystalline state exists between the boundaries of the solid crystalline phase and the isotropic liquid phase (i.e. an intermediate between the three dimensionally ordered crystalline state and the disordered dissolved state). In this state, some of the molecular order characteristics of the solid crystalline phase are retained in the liquid state because of the molecular structure and short range intermolecular interaction. The ability of some compounds to form a liquid crystalline mesophase had been observed nearly a century ago.

Liquid crystals are also known as anisotropic fluids, a fourth state of matter, polymer association structure or mesophases. Those terms are used interchangeably. Lyotropic means a material is formed through changes in solution behavior (and hence by definition contains a solvent, for example water) of the ingredients. The changes involve thermal and solvation energies. The term “lyotropic liquid crystal” as used herein, refers to a liquid crystalline phase distinctive by the presence of birefringence (a non-limiting example of which is formation of maltese crosses) under polarized light microscopy. These are most easily observed in the absence of particles as some particles also demonstrate birefringence. In addition, the term “polymer liquid crystals”, as used herein, means “polymeric lyotropic liquid crystals” unless otherwise specified.

In general, liquid phases refer to the manner in which molecules, in this case cationic polymers and the anionic detersive surfactants, are arranged in space within a phase (this case involves a continuous aqueous phase). This phase is significantly more ordered than an ordinary liquid, but significantly less ordered than crystalline solids. If we consider a crystalline solid to have order in all directions, X, Y and Z then liquid crystals are phases that are ordered or crystalline in only one or two of their three possible orthogonal directions and are disordered (random or liquid-like) in the other dimensions. Cross-linked polymers have the back bones of the polymers chemically bound to each other. This forms a 3-dimensional polymer structure and without being bound by theory, the desired lytropic liquid crystal consist as layers of polymer and surfactant and hence the polymer needs a certain degree of flexibility to form the liquid crystal phase. The inflexibility of the cross-linked polymer is therefore not preferred. Reference: Chapter 8 “The Aqueous Phase Behavior of Surfactants” by R. G. Laughlin. Lamellar liquid crystals are ordered in only the Z direction perpendicular to the plane of the layers and disordered in the X & Y directions within the plane of the layers. Preferably, lamellar liquid crystals are formed in the cleansing composition of the present invention and incorporate non-crosslinked cationic polymers.

Liquid crystals are substances that possess mechanical properties resembling those of fluids, yet are capable of transmitting light when viewed with cross polars (birefringence) under static conditions. Some cases may show Bragg reflections characteristic of a well-defined molecular spacing. They have high degrees of orientational order and chain extensions.

The light microscopy of liquid crystals is described in The Microscopy of Liquid Crystals, Norman Hartshorne, Microscopy Publications, Ltd., Chicago, Ill., U.S.A., 1974. Birefringence occurs in general for mesomorphic states. Methods for microscopic observation and evaluation are discussed in Chapter 1, pp. 1-20, and in Chapter 6, pp. 79-90. A preferred method for determining occurrence of liquid crystals is by observing birefringence (a non-limiting example of which is formation of maltese crosses) of thin liquid crystal films between glass slides or from thin slices of a material under a polarizing microscope.

A further embodiment of the present invention concerns the surprising discovery that particular types of polymeric liquid crystals yield improved conditioning, even without the presence of any additional conditioning agents. Without being limited by theory, these correspond to large and/or more viscous polymeric liquid crystals. The following table exemplifies several of the highly preferred polymers and their liquid crystal size and their rheological property as measured by the storage modulus G′.

The liquid crystal size was measured via standard polarized light microscopy and the size reported as a range based on a finite number of observations. The observed size depends greatly on the preparation technique (for example, the amount of shear in making the cleansing composition) and the following data were measured using a standardized making procedure. The rheological property G′ is defined as the storage modulus and is the part of the shear stress that is in phase with the (shear) strain divided by the strain under sinusoidal conditions. The units of measure are Pa. Additional information may be obtained from “An Introduction to Rheology” by Barnes et al., Elsevier, 1998, incorporated herein by reference.

| | | Polymeric Liquid | |
|---|---|---|---|
| | | Crystal size | G′ of Polymeric |
| | MW | microns | liquid crystal |
| MAPTAC (0) | 220,000 | 5-7 | 500 |
| HMW MAPTAC (1) | 860,000 | 7-9 | 1000 |
| HHMW MAPTAC (2) | 1,500,000 | 8-10 | 1500 |
| Diquat (3) | 900,000 | 9-11 | 750 |

(0), (1), (2) 1-Propanaminium, N,N,N-trimethyl-3-[(2-methyl-1-oxo-2-propenyl)amino]-, chloride; (Poly(Methacrylamidopropyl trimethyl ammonium chloride))
(3) Methacryloamidopropyl-pentamethyl-1,3-propylene-2-ol-ammonium dichloride

These data refer to homopolymers of synthetic cationic polymers, and clearly demonstrate that synthetic cationic polymers with higher molecular weight yield larger polymeric liquid crystals sizes and liquid crystals with higher values of G′.

The inventors have discovered that production of such type of polymeric liquid crystal can be achieved through utilizing the polymers with the following characteristics:
a. a cationic charge density of from about 2 meq/gm to about 10 meq/gm, or
b. an average molecular weight of at least 500,000, or
c. A copolymer formed from one or more cationic monomer units and one or more nonionic monomer units or monomer units bearing a terminal negative charge wherein the subsequent charge of the copolymer is positive.

A further objective of the present invention is to deposit efficacious levels of dispersed conditioning agent particles.

In an embodiment of the present invention, the particulate zinc material has a specified zinc lability within a surfactant system. Zinc lability is a measure of the chemical availability of zinc ion. Soluble zinc salts that do not complex with other species in solution have a relative zinc lability, by definition, of 100%. The use of partially soluble forms of zinc salts and/or incorporation in a matrix with potential complexants generally lowers the zinc lability substantially below the defined 100% maximum.

Labile zinc is maintained by choice of an effective particulate zinc material or formation of an effective particulate zinc material in-situ by known methods.

It has now surprisingly been found, in accordance with the present invention, that anti-dandruff efficacy can be dramatically increased in topical compositions by the use of polyvalent metal salts of pyrithione, such as zinc pyrithione, in combination with particulate zinc materials, and further provide improved conditioning benefits. Therefore an embodiment of the present invention provides topical compositions with improved benefits to the skin and scalp (e.g., improved antidandruff efficacy and improved conditioning).

An embodiment of the present invention provides a stable composition for particulate zinc material dispersion where the zinc source resides in a particulate form. It has been shown to be challenging to formulate aqueous systems containing a particulate zinc material, due to the particulate zinc material's unique physical and chemical properties. Particulate zinc material may have a high density (approximately 3 g/cm³), and needs to be evenly dispersed throughout the product and so it will not aggregate or settle. Particulate zinc material also has a very-reactive surface chemistry as well as the propensity to dissolve in systems with pH values below 6.5. Further, it has been surprisingly found in order for the particulate zinc material will remain as labile, in the presence of a surfactant with an anionic functional group.

A particulate zinc material with a solubility of less than 25% will have a measurable % soluble zinc value below a threshold value determined by the weight percent and molecular weight of the zinc compound. The theoretical threshold value can be calculated by the following equation:

$$\frac{\begin{matrix}0.25 * wt . \% Zn Compound in Composition * \\ molesof Zincin Compound * 65.39 ( MW of Zn )\end{matrix}}{MW of Zn Compound}$$

An embodiment of the present invention is directed to a composition an effective amount of a particulate zinc material; an effective amount of a surfactant including a surfactant with an anionic functional group; an effective amount of a pyrithione or a polyvalent metal salt of a pyrithione; from about 0.025% to about 5% by weight of a water soluble or dispersible, cationic, non-crosslinked, conditioning homopolymer or copolymer having a cationic charge density of from about 2 meq/gm to about 10 meq/gm; and from about 20% to about 95% of an aqueous carrier, by weight of said composition.

An embodiment of the present invention provides topical skin and/or hair compositions which provide superior benefits from particulate zinc material. An embodiment of the present invention also provides a method for cleansing the hair and/or skin. These, and other benefits, will become readily apparent from the detailed description.

The present invention can comprise, consist of, or consist essentially of the essential elements and limitations of the invention described herein, as well any of the additional or optional ingredients, components, or limitations described herein.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

The components and/or steps, including those, which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term “charge density”, as used herein, refers to the ratio of the number of positive charges on a monomeric unit of which a polymer is comprised to the molecular weight of said monomeric unit. The charge density multiplied by the polymer molecular weight determines the number of positively charged sites on a given polymer chain.

The term “fluid” as used herein, means a liquid or a gas which tends to take the shape of its container, container being the wall of the flexible hollow particles.

The term “lamellar liquid crystal” as used herein, means a material that is ordered in only the Z direction perpendicular to the plane of the layers and disordered in the X & Y directions within the plane of the layers.

The term “liquid crystal” as used herein, means a material having phases that are ordered and/or crystalline in only one or two of their three possible orthogonal directions and are disordered (random and/or liquid-like) in the other dimensions.

The term “lyotropic” as used herein, means a material is formed through changes in solution behavior of the ingredients. The changes involve thermal and solvation energies.

The term “phase separation” as used herein, means the formation of two thermodynamically stable liquid phases which exist, not as distinct bulk layers, but as a stable emulsion comprising droplets of one phase dispersed in another phase.

The term “polymer” as used herein shall include materials whether made by polymerization of one type of monomer or made by two (i.e., copolymers) or more types of monomers.

The term “shampoo” as used herein means a composition for cleansing and conditioning hair or skin, including scalp, face, and body.

The term “suitable for application to human hair” as used herein, means that the compositions or components thereof so described are suitable for use in contact with human hair and the scalp and skin without undue toxicity, incompatibility, instability, allergic response, and the like.

The term “water soluble” as used herein, means that the polymer is soluble in water in the present composition. In general, the polymer should be soluble at 25° C. at a concentration of 0.1% by weight of the water solvent, preferably at 1%, more preferably at 5%, more preferably at 15%.

As used herein, “nonvolatile” refers to any material having little or no significant vapor pressure under ambient conditions, and a boiling point under one atmosphere (atm) preferably at least about 250° C. The vapor pressure under such conditions is preferably less than about 0.2 mm.

### A. PARTICULATE ZINC MATERIAL

The composition of the present invention includes an effective amount of a particulate zinc material. Preferred embodiments of the present invention include from about 0.001% to about 10% of a particulate zinc layered material; more preferably from about 0.01% to about 7%; more preferably still from about 0.1% to about 5%.

Particulate zinc materials (PZM's) are zinc-containing materials which remain mostly insoluble within formulated compositions. Many benefits of PZM's require the zinc ion to be chemically available without being soluble, this is termed zinc lability. Physical properties of the particulate material have the potential to impact lability. We have discovered several factors which impact zinc lability and therefore have led to development of more effective formulas based on PZM's.

Particle physical properties which have been found to be important to optimize zinc lability of PZM's are morphology of the particle, surface area, crystallinity, bulk density, surface charge, refractive index, and purity level and mixtures thereof. Control of these physical properties has been shown to increase product performance.

Examples of particulate zinc materials useful in certain embodiments of the present invention include the following:

Inorganic Materials: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

Further, layered structures are those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLM's) may have zinc incorporated in the layers and/or as more labile components of the gallery ions.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLM's, which are often, but not always, synthetic, is layered doubly hydroxides, which are generally represented by the formula [M²⁺₁₋ₓM³⁺ₓ(OH)₂]^{x+}A^{m-}_{x/m}-nH₂O and some or all of the divalent ions (M²⁺) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1=3y)/n}-nH₂O where the two metal ion may be different; if they are the same and represented by zinc, the formula simplifies to [Zn₁₊ₓ(OH)₂]^{2x+2}×A⁻.nH₂O. This latter formula represents (where x=0.4) common materials such as zinc hydroxychloride and zinc hydroxynitrate. These are related to hydrozincite as well wherein the divalent anion is replaced by a monovalent anion. These materials can also be formed in situ in a composition or in or during a production process.

These classes of ZLM's represent relatively common examples of the general category and are not intended to be limiting as to the broader scope of materials which fit this definition.

Natural Zinc containing materials/Ores and Minerals: Sphalerite (zinc blende), Wurtzite, Smithsonite, Franklinite, Zincite, Willemite, Troostite, Hemimorphite and combinations thereof.

Organic Salts: Zinc fatty acid salts (i.e., caproate, laurate, oleate, stearate, etc.), Zinc salts of alkyl sulfonic acids, Zinc naphthenate, Zinc tartrate, Zinc tannate, Zinc phytate, Zinc monoglycerolate, Zinc allantoinate, Zinc urate, Zinc amino acid salts (i.e., methionate, phenylalinate, tryptophanate, cysteinate, etc) and combinations thereof.

Polymeric Salts: Zinc polycarboxylates (i.e., polyacrylate), Zinc polysulfate and combinations thereof.

Physically Adsorbed Forms: Zinc-loaded ion exchange resins, Zinc adsorbed on particle surfaces, Composite particles in which zinc salts are incorporated, (i.e., as core/shell or aggregate morphologies) and combinations thereof.

Zinc Salts: zinc oxalate, zinc tannate, zinc tartrate, zinc citrate, zinc oxide, zinc carbonate, zinc hydroxide, zinc oleate, zinc phosphate, zinc silicate, zinc stearate, zinc sulfide, zinc undecylate, and the like, and mixtures thereof; preferably zinc oxide or zinc carbonate basic.

Commercially available sources of zinc oxide include Z-Cote and Z-Cote HPI (BASF), and USP I and USP II (Zinc Corporation of America).

Commercially available sources of zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

### Particle Size of PZM

In an embodiment of the present invention, it is has been found that a smaller particle size is inversely proportional to relative zinc lability

D(90) is the particle size which corresponds to 90% of the amount of particles are below this size. In an embodiment of the present invention, the particulate zinc material may have a particle size distribution wherein 90% of the particles are less than about 50 microns. In a further embodiment of the present invention, the particulate zinc material may have a particle size distribution wherein 90% of the particles are less than about 30 microns. In yet a further embodiment of the present invention, the particulate zinc material may have a particle size distribution wherein 90% of the particles are less than about 20 microns.

### Surface Area of PZM

In an embodiment of the present invention, there may be a direct relationship between surface area and relative zinc lability.

Increased particle surface area generally increases zinc lability due to kinetic factors. Particulate surface area can be increased by decreasing particle size and/or altering the particle morphology to result in a porous particle or one whose overall shape deviates geometrically from sphericity.

In an embodiment of the present invention, the basic zinc carbonate may have a surface area of greater than about 10 m²/gm. In a further embodiment, the basic zinc carbonate may have a surface area of greater than about 20 m²/gm. In yet a further embodiment of the present invention, the basic zinc carbonate may have a surface area of greater than about 30 m²/gm.

### Zinc Binding Materials

Materials which have a high affinity for zinc and have the tendency to result in the formation of insoluble complexes of zinc can foul the surface of particulate zinc materials (PZM's). By “fouling” it is meant the formation of an insoluble surface layer of the zinc binding material (ZBM) zinc salt which interferes with the kinetic lability of zinc from the base PZM material. The magnitude of negative effect of ZBM's is the product of the strength of association to zinc and the relative amount of the ZBM (relative to the PZM surface area). The PZM's can tolerate a portion of surface coverage without substantial inhibition of kinetic lability.

Those materials with high potential to bind to the PZM surface are ZBM's that form only sparingly soluble salts with zinc in water. “Sparingly soluble” refers to zinc salts with 1 gram(g)/100 g water solubility or less. These are the materials that form precipitated surface species on the PZM that interfere with zinc lability. Some non-limiting examples of zinc binding materials are laurate, citrate, valerate, oxalate, tartrate, iodate, thiocyanate, cyanide, sulfide, pyrophosphate, phosphate and mixtures thereof. A summary of the solubilities of common zinc salts and further disclosure of zinc biding material is found in U.S. application Ser. No. 11/216,520, filed Aug. 31, 2005 on pages 9-12 and incorporated by reference herein. Many common raw materials may be sources for inadvertent ZBM's. In the case of fatty acids, for example, any material which originates from triglycerides or fatty acids will likely contain some level of fatty acid ZBM in the raw material as used. Surfactants derived directly from triglycerides or those derived from fatty alcohols which are themselves derived from triglycerides will contain varying levels of fatty acids. Other raw materials may contain relatively low levels of ZBM's that are added for a secondary benefit. For example, citric acid is commonly used for pH control during raw material manufacture. It is not always obvious to the end user of a raw material if such ZBM's are present; this information can be obtained from the manufacturer or analyzed directly.

Maximization of zinc lability from PZM's requires either complete avoidance of the presence of ZBM's or limiting the amount of the material to avoid complete coverage of the surface area of the PZM (i.e., saturation). An approximation of the amount of ZBM required to completely cover a PZM can be calculated based on effective surface area of the PZM and a knowledge of how tightly the ZBM can pack on the surface. The following example is illustrative of the process of approximating how much ZBM is certain to saturate and foul the entire PZM surface. It will be calculated for the general case in which a ZBM packs on the surface in a manner analogous to a surfactant adsorbing at an oil-water interface. In this case, a common value for surface area occupied per molecule is 30 Å² (equivalent to 3×10⁻⁷μ²). It will be calculated per gram of a PZM with a measured surface area (SA, in m²/g):

$$\frac{1 {m}^{2} ZBM}{g PZM \cdot SAPZM} \times \frac{ZBM molecule}{3 \times {10}^{- 7} {µ}^{2} ZBM} \times {( \frac{1 \times {10}^{6} µ ZBM}{m ZBM} )}^{2} \times \frac{mol ZBM}{6.02 \times {10}^{23} molecules ZBM} \times \frac{1 \times {10}^{6} µ mol ZBM}{mol ZBM} = 5.5 µ mol ZBM \text{/} g PZM - SA PZM$$

Thus, 5.5 micromoles of ZBM will saturate 1 g of a PZM with a surface area of 1 m²/g. Therefore, for the present invention, it is desirable that the composition comprises less than 5.5 micromoles of a zinc binding material (ZBM) per gram (g) of a particulate zinc material (PZM)/per m²/g surface area of a particulate zinc material (PZM).
For an example of zinc carbonate (a PZM) with a surface area of 30 m²/g and laurate as the ZBM, the calculation then becomes:

$$\frac{30 {m}^{2} ZC}{g ZC} \times {( \frac{1 \times {10}^{6} µ ZC}{m ZC} )}^{2} \times \frac{LA molecule}{3 \times {10}^{- 7} {µ}^{2}} \times \frac{mole LA}{6.02 \times {10}^{23} molecules LA} \times \frac{200 g LA}{mole LA} = 0.03 g LA \text{/} g ZC$$

Thus, approximately 0.03 g of laurate would saturate and foul the surface of one gram of a zinc carbonate PZM with the specified surface area. Based on this type of analysis, other “fouling levels” can be established for the specific ZBM-PZM combination. However, this example provides an approximation of the range of levels that need to be controlled to assure zinc lability of the PZM.

More specifically, then, a formulation containing 1.6% of the zinc carbonate specified above would require a laurate level below 0.048% (480 ppm) to remain effective. This would represent the total laurate present, whether added directly or inadvertently entering a formula via other raw material additions. This level also assumes there are no other ZBM's present; if there are, each needs to be considered separately while maintaining a combined amount below surface saturation level.

### B. PYRITHIONE OR A POLYVALENT METAL SALT OF PYRITHIONE

In a preferred embodiment, the present may comprise pyrithione or a polyvalent metal salt of pyrithione. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

It is further contemplated that when ZPT is used as the anti-microbial particulate in the anti-microbial compositions herein, that an additional benefit of hair growth or re-growth may be stimulated or regulated, or both, or that hair loss may be reduced or inhibited, or that hair may appear thicker or fuller.

Zinc pyrithione may be made by reacting 1-hydroxy-2-pyridinethione (i.e., pyrithione acid) or a soluble salt thereof with a zinc salt (e.g. zinc sulfate) to form a zinc pyrithione precipitate, as illustrated in U.S. Pat. No. 2,809,971.

Preferred embodiments include from about 0.01% to about 5% of a pyrithione or polyvalent metal salt of a pyrithione; more preferably from about 0.1% to about 2%.

In embodiments having a particulate zinc material and a pyrithione or polyvalent metal salt of pyrithione,

the ratio of particulate zinc material to pyrithione or a polyvalent metal salt of pyrithione is preferably from 5:100 to 10:1; more preferably from about 2:10 to 5:1; more preferably still from 1:2 to 3:1.

### C. TOPICAL CARRIER

In a preferred embodiment, the composition of the present invention is in the form of a topical composition, which includes a topical carrier. Preferably, the topical carrier is selected from a broad range of traditional personal care carriers depending on the type of composition to be formed. By suitable selections of compatible carriers, it is contemplated that such a composition is prepared in the form of daily skin or hair products including conditioning treatments, cleansing products, such as hair and/or scalp shampoos, body washes, hand cleansers, water-less hand sanitizer/cleansers, facial cleansers and the like.

In a preferred embodiment, the carrier is water. Preferably the compositions of the present invention comprise from 40% to 95% water by weight of the composition; preferably from 50% to 85%, more preferably still from 60% to 80%.

### D. DETERSIVE SURFACTANT

The composition of the present invention includes a detersive surfactant. The detersive surfactant component is included to provide cleaning performance to the composition. The detersive surfactant component in turn comprises anionic detersive surfactant, zwitterionic or amphoteric detersive surfactant, or a combination thereof. Such surfactants should be physically and chemically compatible with the essential components described herein, or should not otherwise unduly impair product stability, aesthetics or performance.

Suitable anionic detersive surfactant components for use in the composition herein include those which are known for use in hair care or other personal care cleansing compositions. The concentration of the anionic surfactant component in the composition should be sufficient to provide the desired cleaning and lather performance, and generally range from about 2% to about 50%, preferably from about 8% to about 30%, more preferably from about 10% to about 25%, even more preferably from about 12% to about 22%.

Preferred anionic surfactants suitable for use in the compositions are the alkyl and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from 1 to 10, and M is a cation such as ammonium, alkanolamines, such as triethanolamine, monovalent metals, such as sodium and potassium, and polyvalent metal cations, such as magnesium, and calcium.

Preferably, R has from about 8 to about 18 carbon atoms, more preferably from about 10 to about 16 carbon atoms, even more preferably from about 12 to about 14 carbon atoms, in both the alkyl and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. Lauryl alcohol and straight chain alcohols derived from coconut oil or palm kernel oil are preferred. Such alcohols are reacted with between about 0 and about 10, preferably from about 2 to about 5, more preferably about 3, molar proportions of ethylene oxide, and the resulting mixture of molecular species having, for example, an average of 3 moles of ethylene oxide per mole of alcohol, is sulfated and neutralized.

Other suitable anionic detersive surfactants are the water-soluble salts of organic, sulfuric acid reaction products conforming to the formula [R¹—SO₃-M] where R¹ is a straight or branched chain, saturated, aliphatic hydrocarbon radical having from about 8 to about 24, preferably about 10 to about 18, carbon atoms; and M is a cation described hereinbefore.

Still other suitable anionic detersive surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic detersive surfactants suitable for use in the compositions are the succinnates, examples of which include disodium N-octadecylsulfosuccinnate; disodium lauryl sulfosuccinate; diammonium lauryl; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic detersive surfactants include olefin sulfonates having about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non limiting example of such an alpha-olefin sulfonate mixture is described in U.S. Pat. No. 3,332,880.

Another class of anionic detersive surfactants suitable for use in the compositions are the beta-alkyloxy alkane sulfonates. These surfactants conform to the formula

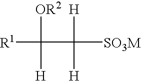

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore.

Preferred anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment of the present invention, the anionic surfactant is preferably sodium lauryl sulfate or sodium laureth sulfate.

Suitable amphoteric or zwitterionic detersive surfactants for use in the composition herein include those which are known for use in hair care or other personal care cleansing. Concentration of such amphoteric detersive surfactants preferably ranges from about 0.5% to about 20%, preferably from about 1% to about 10%. Non limiting examples of suitable zwitterionic or amphoteric surfactants are described in U.S. Pat. Nos. 5,104,646 (Bolich Jr. et al.), 5,106,609 (Bolich Jr. et al.).

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Preferred amphoteric detersive surfactants for use in the present invention include cocoamphoacetate, cocoamphodiacetate, lauroamphoacetate, lauroamphodiacetate, and mixtures thereof.

Zwitterionic detersive surfactants suitable for use in the composition are well known in the art, and include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines are preferred.

The compositions of the present invention may further comprise additional surfactants for use in combination with the anionic detersive surfactant component described hereinbefore. Suitable optional surfactants include nonionic and cationic surfactants. Any such surfactant known in the art for use in hair or personal care products may be used, provided that the optional additional surfactant is also chemically and physically compatible with the essential components of the composition, or does not otherwise unduly impair product performance, aesthetics or stability. The concentration of the optional additional surfactants in the composition may vary with the cleansing or lather performance desired, the optional surfactant selected, the desired product concentration, the presence of other components in the composition, and other factors well known in the art.

Non limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378.

### E. DISPERSED PARTICLES

The composition of the present invention may include dispersed particles. In the compositions of the present invention, it is preferable to incorporate at least 0.025% by weight of the dispersed particles, more preferably at least 0.05%, still more preferably at least 0.1%, even more preferably at least 0.25%, and yet more preferably at least 0.5% by weight of the dispersed particles. In one embodiment of the present invention, particles useful can be inorganic, synthetic, or semi-synthetic in origin. In the compositions of the present invention, it is preferable to incorporate no more than about 20% by weight of the dispersed particles, more preferably no more than about 10%, still more preferably no more than 5%, even more preferably no more than 3%, and yet more preferably no more than 2% by weight of the dispersed particles.

One embodiment of the present invention comprises dispersed, water insoluble solid particles. Without being bound by theory, it is believed that the dispersed, water insoluble solid particles may partition into the microscopically-phase separate lyotropic liquid crystals formed in an embodiment of the present invention.

In this embodiment of the present invention the solid particles preferably have a particle size of less than 300 μm. Typically, the particles will have a particle size from about 0.01 μm to about 80 μm, still more preferably from about 0.1 μm to about 70 μm, also more preferably from about 0.5 μm to about 65 μm, and even more preferably from about 1 μm to about 60 μm in diameter. The solid particles of the present invention preferably have an average particle size of less than 300 μm. Typically, the particles will have an average particle size from about 0.01 μm to about 80 μm, still more preferably from about 0.1 μm to about 70 μm, also more preferably from about 0.5 μm to about 65 μm, and even more preferably from about 1 μm to about 60 μm in diameter.

Typical solid particle levels are selected for the particular purpose of the composition. As example, where it is desired to deliver color benefits, pigment solid particles confering the desired hues can be incorporated. Where hair volume or style retention benefits are desired, solid particles capable of conferring friction can be used to reduce disruption and collapse of the hair style. Where conditioning or slip is desired, suitable platelet or spherical solid particles can be incorporated. Where anti-dandruff treatment is desired, suitable anti-dandruff solid particles such as zinc pyrithione can be incorporated for a desired zinc pyrithione deposition. Determination of the levels and solid particle types is within the skill of the artisan solid particles that are generally recognized as safe, and are listed in C.T.F.A. Cosmetic Ingredient Handbook, Sixth Ed., Cosmetic and Fragrance Assn., Inc., Washington D.C. (1995), incorporated herein by reference, can be used.

Solid particles useful in the present invention can be inorganic, synthetic, or semi-synthetic in composition. Hybrid solid particles are also useful. Synthetic solid particles can be made of either cross-linked or non cross-linked polymers. The solid particles of the present invention can have surface charges or their surface can be modified with organic or inorganic materials such as surfactants, polymers, and inorganic materials. Solid particle complexes are also useful.

Non limiting examples of inorganic solid particles include various silica particles including colloidal silicas, fumed silicas, precipitated silicas and silica gels. Non-limiting examples of synthetic solid particles include nylon, silicone resins, poly(meth)acrylates, polyethylene, polyester, polypropylene, polystyrene, polyurethane, polyamide, epoxy resins, urea resins, and acrylic powders.

Non limiting examples of other dispersed, water insoluble solid particles for use in the compositions of the present invention are described in U.S. Pat. No. 6,849,584 which descriptions are incorporated herein by reference.

### F. SYNTHETIC CATIONIC POLYMER

In an embodiment of the present invention, the shampoo compositions comprise certain cationic deposition or conditioning polymers that, in combination with the anionic surfactant component and other essential components herein, form polymeric liquid crystals. The polymers can be formulated in a stable shampoo composition that provides improved conditioning performance when formulated without additional conditioning actives, and also provides improved deposition of the conditioning agent particles (described herein) onto hair. The cationic synthetic polymer may be formed from

i) one or more cationic monomer units, and optionally

ii) one or more monomer units bearing a terminal negative charge, and/or

iii) a functional nonionic monomer,

wherein the subsequent charge of the copolymer is positive. The ratio of the three types of monomers is given by m, p and q where m is the number of cationic monomers, p the number of monomers bearing a terminal negative charge and q is the number of functional nonionic monomers.

The synthetic cationic polymers suitable for use in the shampoo composition herein are water soluble or dispersible, non crosslinked, cationic polymers having the following structure:

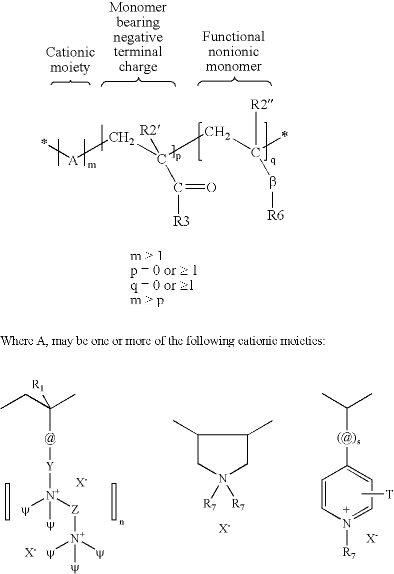

Where @=amido, alkylamido, ester, ether, alkyl or alkylaryl.
Where Y=C1-C22 alkyl, alkoxy, alkylidene, alkyl or aryloxy
Where ψ32 C1-C22 alkyl, alkyloxy, alkyl aryl or alkyl aryloxy
Where Z=C1-C22 alkyl, alkyloxy, aryl or aryloxy
Where R1=H, C1-C4 linear or branched alkyl
Where s=0 or 1, n=0 or ≧1
Where T and R7=C1-C22 alkyl
Where X⁻=halogen, hydroxide, alkoxide, sulfate or alkylsulfate

Examples of cationic monomers consist of aminoalkyl(meth)acrylates, (meth)aminoalkyl(meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl(meth)acrylate, ditertiobutylaminoethyl (meth)acrylate, dimethylaminomethyl(meth)acrylamide, dimethylaminopropyl (meth)acrylamide; ethylenimine, vinylamine, 2-vinylpyridine, 4-vinylpyridine; trimethylammonium ethyl(meth)acrylate chloride, trimethylammonium ethyl (meth)acrylate methyl sulphate, dimethylammonium ethyl(meth)acrylate benzyl chloride, 4-benzoylbenzyl dimethylammonium ethyl acrylate chloride, trimethyl ammonium ethyl (meth)acrylamido chloride, trimethyl ammonium propyl(meth)acrylamido chloride, vinylbenzyl trimethyl ammonium chloride, diallyldimethyl ammonium chloride.

Preferred cationic monomers comprise quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chloride and bromides, sulphates, hydrosulphates, alkylsulphates (for example comprising 1 to 6 carbon atoms), phosphates, citrates, formates, and acetates.

Preferred cationic monomers include trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride.

More preferred cationic monomers include trimethyl ammonium propyl (meth)acrylamido chloride.

Where the monomer bearing a terminal negative charge is defined by R2′=H, C₁-C₄ linear or branched alkyl and R3 as:

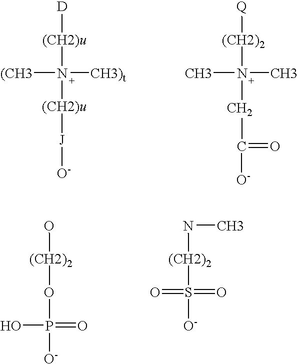

Where D=electronegative element chosen between oxygen, nitrogen, sulfur

### Where Q=NH2 or O

Where u=1-6
Where t=0-1
J=oxygenated functional group containing the following elements P, S, C Examples of monomers bearing a terminal negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Preferred monomers with a terminal negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic

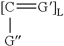

acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS). Where the functional nonionic monomer is defined by R2″=H, C₁-C₄ linear or branched alkyl, R6=linear or branched alkyl, alkyl aryl, aryl oxy, alkyloxy, alkylaryl oxy and P is defined as

### Where G′ and G″=O, S or N—H and L=0 or 1.

Examples of such nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Preferred nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The concentration of the cationic polymer in the shampoo composition ranges about 0.025% to about 5%, preferably from about 0.1% to about 3%, more preferably from about 0.2% to about 1%, by weight of the composition.

The anionic counterion (X⁻) in association with the cationic conditioning polymers may be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non limiting examples of such counterions include halides (e.g., chlorine, fluorine, bromine, iodine), sulfate and methylsulfate.

### Homopolymer

The cationic polymer, by definition must contain cationic monomers and hence m must be greater than 1. However, in the case of homopolymers, there is only cationic monomers and hence p and q are zero. The homopolymer has either a cationic charge density of from about 2 meq/gm to about 10 meq/gm or an average molecular weight of at from about 1000 to 5 million.

Highly preferred synthetic cationic hompolymers have high charge densities of from about 3 meq/gm to about 10 meq/gm, even further preferred from about 4 meq/gm to about 7 meq/gm. Other highly preferred synthetic cationic homopolymers have high charge densities of from about 8 meq/gm to about 10 meq/gm. The following structures are highly preferred synthetic cationic homopolymers. In the case of R1=CH3, the charge density is 5.60 when n=1.

(Methacryloamidopropyl-pentamethyl-1,3-propylene-2-ol-ammonium dichloride), in the case or R1=CH3, the charge density is 6.07 when n=2 (N,N,N,N′,N′,N″,N″-heptamethyl-N″-3-(1-oxo-2-methyl-2-propenyl)aminopropyl-9-oxo-8-azo-decane-1,4,10-triammonium trichloride.) and in the case of R1=H, the charge density is 4.88 when n=0.

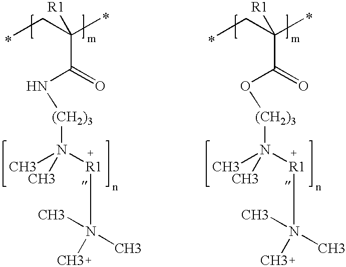

Another class of highly preferred homopolymers have an average molecular weight of about 1000 to about 5 million, preferably from about 10,000 to about 2,000,000. A highly preferred homopolymer conforms to the following structure:

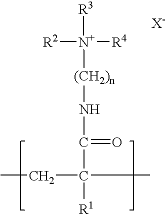

wherein R¹ is hydrogen, methyl or ethyl; each of R², R³ and R⁴ are independently hydrogen or a short chain alkyl having from about 1 to about 8 carbon atoms, preferably from about 1 to about 5 carbon atoms, more preferably from about 1 to about 2 carbon atoms; n is an integer having a value of from about 1 to about 8, preferably from about 1 to about 4; and X is a counterion. The nitrogen attached to R², R³ and R⁴ may be a protonated amine (primary, secondary or tertiary), but is preferably a quaternary ammonium wherein each of R², R³ and R⁴ are alkyl groups.

In an embodiment of the present invention a highly preferred homopolymer conforms to the following structure:

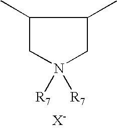

Wherein R7=C1-C22 alkyl
Where X⁻=halogeno, hydroxide, alkoxide, sulfate or alkylsulfate

### Copolymers

The copolymer formed from one or more cationic monomer units and one or more monomer units bearing a terminal negative charge or a functional nonionic monomer, wherein the subsequent charge of the copolymer is positive. In the case of the preferred copolymers p and/or q are greater than 1. In the case that there are monomers with units bearing a terminal negative charge, the overall polymer should be positive in charge and hence m>p.

A highly preferred synthetic cationic copolymer has charge densities of from about 2 meq/gm to about 10 meq/gm, and more preferably from 2 meq/gm to 7 meq/gm.

Another class of highly preferred copolymers has an average molecular weight of about 1,000 to about 5,000,000, preferably from about 100,000 to about 2,000,000

Examples of highly preferred copolymers include:

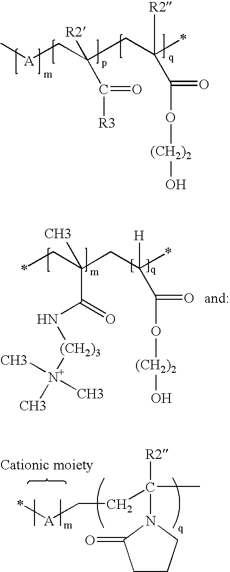

Where A, may be one or more of the following cationic moieties:

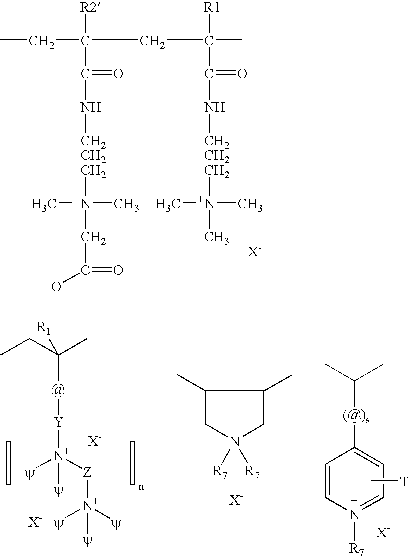

Where @=amido, alkylamido, ester, ether, alkyl or alkylaryl.
Where Y=C1-C22 alkyl, alkoxy, alkylidene, alkyl or aryloxy
Where ψ=C1-C22 alkyl, alkyloxy, alkyl aryl or alkyl aryloxy
Where Z=C1-C22 alkyl, alkyloxy, aryl or aryloxy
Where R1=H, C1-C4 linear or branched alkyl
Where s=0 or 1, n=0 or ≧1
Where T and R7=C1-C22 alkyl
Where X⁻=halogeno, hydroxide, alkoxide, sulfate or alkylsulfate

Examples of cationic monomers consist of aminoalkyl(meth)acrylates, (meth)aminoalkyl(meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl(meth)acrylate, ditertiobutylaminoethyl (meth)acrylate, dimethylaminomethyl(meth)acrylamide, dimethylaminopropyl (meth)acrylamide; ethylenimine, vinylamine, 2-vinylpyridine, 4-vinylpyridine; trimethylammonium ethyl(meth)acrylate chloride, trimethylammonium ethyl (meth)acrylate methyl sulphate, dimethylammonium ethyl(meth)acrylate benzyl chloride, 4-benzoylbenzyl dimethylammonium ethyl acrylate chloride, trimethyl ammonium ethyl (meth)acrylamido chloride, trimethyl ammonium propyl(meth)acrylamido chloride, vinylbenzyl trimethyl ammonium chloride, diallyldimethyl ammonium chloride.

Preferred cationic monomers comprise quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chloride and bromides, sulphates, hydrosulphates, alkylsulphates (for example comprising 1 to 6 carbon atoms), phosphates, citrates, formates, and acetates.

Preferred cationic monomers include trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl(meth)acrylamido chloride, vinylbenzyl trimethyl ammonium chloride.

More preferred cationic monomers include trimethyl ammonium propyl (meth)acrylamido chloride.

Where the monomer bearing a terminal negative charge is defined by R2′=H, C1-C4 linear or branched alkyl and R3 as:

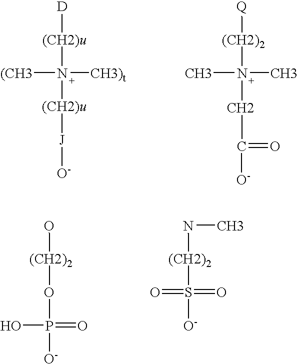

Where D=electronegative element chosen between oxygen, nitrogen, sulfur

### Where Q=NH2 or O

Where u=1-6
Where t=0-1
J=oxygenated functional group containing the following elements P, S, C

Examples of monomers bearing a terminal negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Preferred monomers with a terminal negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS). Where the functional nonionic monomer is defined by R2″=H, C1-C4 linear or branched alkyl, R6=linear or branched alkyl, alkyl aryl, aryl oxy, alkyloxy, alkylaryl oxy and V is defined as

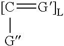

### Where G′ and G″=O, S or N—H and L=0 or 1.

Examples of such nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitrites, vinylamine amides, vinyl alcohol, vinyl pyrolidone, and vinyl aromatic compounds.

Preferred nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

### F. AQUEOUS CARRIER

The compositions of the present invention are typically in the form of pourable liquids (under ambient conditions). The compositions will therefore typically comprise an aqueous carrier, which is present at a level of from about 20% to about 95%, preferably from about 60% to about 85%. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

### G. ADDITIONAL COMPONENTS

The compositions of the present invention may further comprise one or more optional components known for use in hair care or personal care products, provided that the optional components are physically and chemically compatible with the essential components described herein, or do not otherwise unduly impair product stability, aesthetics or performance. Individual concentrations of such optional components may range from about 0.001% to about 10%.

Non-limiting examples of optional components for use in the composition include cationic polymers, conditioning agents (hydrocarbon oils, fatty esters, silicones), anti dandruff agents, suspending agents, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins, minerals, herbal/fruit/food extracts, sphingolipids derivatives or synthetical derivative, and clay.

In a further embodiment of the present invention an additional synthetic cationic polymer may be present. The additional synthetic cationic polymer may have a charge density of less than 2.0 meq/gm. A non-limiting example of such an additional synthetic cationic polymer is AM:TRIQUAT which is a co-polymer between acrylamide and 1,3-Propanediaminium,N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride. Further name for AM:TRIQUAT would be PQ76.

### Naturally Derived Cationic Polymer

In an embodiment of the present invention, the compositions of the present invention may comprise another or an additional polymer, or a second cationic polymer, which is a low charge density cationic polymer. In a preferred embodiment, the further cationic polymer is a naturally derived cationic polymer. The term, “naturally derived cationic polymer” as used herein, refers to cationic polymers which are obtained from natural sources. The natural sources may be polysaccharide polymers. Therefore, the naturally derived cationic polymer may be selected from celluloses, starches, guars, non-guar-galactomannans, and other sources found in nature.

The additional cationic polymer has a molecular weight of from about 1,000 to about 10,000,000, and a cationic charge density of at least about 0.2 meq/g, more preferably at least about 0.4 meq/g, and more preferably at least about 0.5 meq/g. The additional cationic polymer also has a charge density of less than about 4.0 meq/gm, and more preferably less than or equal to about 2 meq/gm. The polymers are typically present in a concentration of from about 0.025% to about 5%, and more preferably from about 0.10% to about 2% by weight of the personal care composition. The additionally cationic polymers form an isotropic coacervate in the neat composition or upon dilution with water. The isotropic coacervate aids in deposition of optional small particle size conditioning agents, and provides excellent wet conditioning performance. Such deposition and wet conditioning enhancement result in hair feel, shine, and other appreciable benefits.

The cationic polymers herein are either soluble in a composition, or are soluble in a complex coacervate phase in a composition formed by the cationic polymer and the anionic detersive surfactant component described hereinbefore. Complex coacervates of the cationic polymer can also be formed with other charged materials, such as anionic polymers, in the composition.

Isotropic coacervate formation is dependent upon a variety of criteria such as molecular weight, component concentration, and ratio of interacting ionic components, ionic strength (including modification of ionic strength, for example, by addition of salts), charge density of the cationic and anionic components, pH, temperature, and the aforementioned surfactant system. Isotropic coacervate systems and the effect of these parameters have been described, for example, by J. Caelles, et al., “Anionic and Cationic Compounds in Mixed Systems”, Cosmetics & Toiletries, Vol. 106, April 1991, pp 49-54, C. J. van Oss, “Coacervation, Complex Coacervation and Flocculation”, J. Dispersion Science and Technology, Vol. 9 (5,6), 1988-89, pp 561-573, and D. J. Burgess, “Practical Analysis of Complex Coacervate Systems”, J. of Colloid and Interface Science, Vol. 140, No. 1, November 1990, pp 227-238.

It is believed to be particularly advantageous for the second cationic polymer to be present in the composition in an isotropic coacervate phase, or to form an isotropic coacervate phase upon application or rinsing of the composition to or from the hair. Complex isotropic coacervates are believed to more readily deposit on the hair than a dissolved polymer. Thus, in general, it is preferred that the first cationic polymer exist in the personal care composition as an isotropic coacervate phase or form an isotropic coacervate phase upon dilution.

Techniques for analysis of formation of complex isotropic coacervates are known in the art. For example, microscopic analyses of the personal care compositions, at any chosen stage of dilution, can be utilized to identify whether an isotropic coacervate phase has formed. Such isotropic coacervate phases will be identifiable as an additional emulsified phase in the composition. The use of dyes can aid in distinguishing the isotropic coacervate phase from other insoluble phases dispersed in the personal care composition.

### Cationic Polysaccharide Polymers

The personal care compositions of the present invention may include a naturally derived cationic polymer which is a cationic polysaccharide polymer. Cationic polysaccharide polymers encompass cellulose polymers, starch polymers, and polymers made up of multiple monosaccharides joined together by glycosidic linkages. Suitable polysaccharide cationic polymers include those which conform to the following formula:

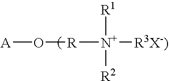

wherein A is an anhydroglucose residual group, such as a cellulose anhydroglucose residual; R is an alkylene oxyalkylene, polyoxyalkylene, or hydroxyalkylene group, or combination thereof; R1, R2, and R3 independently are alkyl, aryl, alkyl-aryl, arylalkyl, alkoxyalkyl, or alkoxyaryl groups, each group containing up to about 18 carbon atoms, and the total No of carbon atoms for each cationic moiety (i.e., the sum of carbon atoms in R1, R2 and R3) preferably being about 20 or less; and X is an anionic counterion. Non-limiting examples of such counterions include halides (e.g., chlorine, fluorine, bromine, iodine), sulfate and methylsulfate. The degree of cationic substitution in these polysaccharide polymers is typically from about 0.01 to about 1 cationic groups per anhydroglucose unit.

In one embodiment of the invention, the cellulose cationic polymers are salts of hydroxyethyl cellulose reacted with trimethyl ammonium substituted epoxide, referred to in the industry (CTFA) as Polyquaternium 10 and available from Amerchol Corp. (Edison, N.J., USA) under the trade name, Ucare Polymer KG-30M, having a cationic charge density of about 1.9 meq/gm.

### Cationically Modified Starch Polymer

The personal care compositions may comprise a naturally derived cationic polymer which is a water-soluble cationically modified starch polymer. As used herein, the term, “cationically modified starch”, refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

A method of chemically modifying the charge densities of the cationically modified starch polymers includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The cationically modified starch polymers may comprise maltodextrin. Thus, in one embodiment of the present invention, the cationically modified starch polymers may be further characterized by a Dextrose Equivalance (“DE”) value of less than about 35, and more preferably from about 1 to about 20. The DE value is a measure of the reducing equivalence of the hydrolyzed starch referenced to dextrose and expressed as a percent (on dry basis). Starch completely hydrolyzed to dextrose has a DE value of 100, and unhydrolyzed starch has a DE value of 0. A suitable assay for DE value includes one described in “Dextrose Equivalent”, Standard Analytical Methods of the Member Companies of the Corn Industries Research Foundation, 1st ed., Method E-26. Additionally, the cationically modified starch polymers may comprise a dextrin. Dextrin is typically a pyrolysis product of starch with a wide range of molecular weights.

The source of starch before chemical modification can be chosen from a variety of sources such as tubers, legumes, cereal, and grains. Non-limiting examples of this source starch may include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof. Waxy corn starch is preferred.

In one embodiment of the present invention, cationically modified starch polymers are selected from degraded cationic maize starch, cationic tapioca, cationic potato starch, and mixtures thereof. In another embodiment, cationically modified starch polymers are cationic corn starch.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers in the present invention may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

Suitable cationically modified starches are available from known starch suppliers, such as National Starch. Also suitable for use in the present invention is nonionic modified starch that could be further derivatized to a cationically modified starch as is known in the art. Other suitable modified starch starting materials may be quaternized, as is known in the art, to produce a cationically modified starch polymer suitable for use in the present invention.

One method of conducting starch degradation involves preparing a starch slurry by mixing granular starch in water. The temperature is raised to about 35° C. An aqueous solution of potassium permanganate is then added at a concentration of about 50 ppm based on starch. The pH is raised to about 11.5 with sodium hydroxide and the slurry is stirred sufficiently to prevent settling of the starch. Then, about a 30% solution of hydrogen peroxide diluted in water is added to a level of about 1% of peroxide based on starch. The pH of about 11.5 is then restored by adding additional sodium hydroxide. The reaction is completed over about a 1 to about 20 hour period. The mixture is then neutralized with dilute hydrochloric acid. The degraded starch is recovered by filtration followed by washing and drying.

### Cationic Galactomannan Polymer

The personal care compositions may comprise a naturally derived cationic polymer which may be a guar or non-guar galactomannan polymer. In one embodiment, the galactomannan polymer is a polymer derivative having a mannose to galactose ratio of 2:1 or greater, on a monomer to monomer basis, and the galactomannan polymer derivative is selected from the group consisting of a cationic galactomannan polymer derivative and an amphoteric galactomannan polymer derivative having a net positive charge. The term “galactomannan polymer derivative”, means a compound obtained from a galactomannan polymer (ie. a galactomannan gum) which is chemically modified. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of μ (1-4) glycosidic linkages. The galactose branching arises by way of an μ (1-6) linkage. The ratio of mannose monomers to galactose monomers varies according to the species of the plant and also is affected by climate. Guar is an example of one type of a galactomannan polymer, specifically having a mannose to galactose ratio of 2 monomers of mannose to 1 monomer of galactose. In one embodiment, the galactomannan polymer derivatives have a ratio of mannose to galactose of greater than 2:1 on a monomer to monomer basis (i.e., non-guar galactomannan polymers). Preferably, the ratio of mannose to galactose is greater than about 3:1, and more preferably the ratio of mannose to galactose is greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to tara gum (3 parts mannose/1 part galactose), locust bean or carob (4 parts mannose/1 part galactose), and cassia gum (5 parts mannose/1 part galactose). Herein, the term “non-guar galactomannan polymer derivatives” refers to cationic polymers which are chemically modified from a non-guar galactomanan polymer. A preferred non-guar galactomannan polymer derivative is cationic cassia and is sold under the trade name Cassia EX-906, which is commercially available from Noveon Inc.

Suitable galactomannan polymer derivatives are described in U.S. Patent Publication No. 2006/0099167A1 to Staudigel et al.

A potential side reaction that may occur during the quaternization reaction of a cationic polymer production process is the formation of trimethylamine (TMA). While not intending to be limited by theory, the presence of TMA as an impurity in a cationic polymer containing composition at a pH greater than 6.8 may be found to be the source of an amine off-odor or fishy off-odor. It has surprisingly been discovered that pH has a significant effect on the level of TMA evolved into the headspace of the composition—in particular, the level of TMA in the headspace increases as the pH increases. Headspace is commonly referred to as the volume above a liquid or solid in a closed container. In turn, the level of amine off-odor can be found to be proportional to the level of TMA present in the headspace. Additionally, it has been discovered that it is possible to reverse the TMA evolution into the headspace by lowering the pH of the composition, as demonstrated and discussed in U.S. application Ser. No. 11/216/520, filed Aug. 31, 2005 on pages 22-26 and incorporated by reference herein.

Therefore, in order to produce an acceptable composition having a pH of greater than 6.8, which comprises a cationic polymer, with low to no amine off-odor, it has been discovered that it may be necessary to use a cationic polymer which contains from no detectable TMA to low levels of TMA. Levels of TMA from a cationic polymer can be measured using the following method as demonstrated and discussed in U.S. application Ser. No. 11/216,520, filed Aug. 31, 2005 on pags 22-26 and incorporated by reference herein.

It has been discovered that compositions comprising cationic polymers which have levels of TMA, as measured, for example, in the method described above, below 45 ppm, preferably below 25 ppm, more preferably below 17 ppm, have no amine off-odor to low amine off-odor which has been found to be acceptable.

### 2. Nonionic Polymers

Polyalkylene glycols having a molecular weight of more than about 1000 are useful herein. Useful are those having the following general formula:

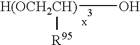

wherein R⁹⁵ is selected from the group consisting of H, methyl, and mixtures thereof. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

### 3. Conditioning Agents

Conditioning agents include any material which is used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The conditioning agents useful in the compositions of the present invention typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

The concentration of the conditioning agent in the composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

1. Silicones

The conditioning agent of the compositions of the present invention is preferably an insoluble silicone conditioning agent. The silicone conditioning agent particles may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent typically ranges from about 0.01% to about 10%, preferably from about 0.1% to about 8%, more preferably from about 0.1% to about 5%, more preferably from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, more preferably from about 100,000 to about 1,500,000 csk.

The dispersed silicone conditioning agent particles typically have a volume average particle diameter ranging from about 0.01 μm to about 50 μm, as measured using the Horiba LA-910 Particle Size Analyzer. The Horiba LA-910 instrument uses the principles of low-angle Fraunhofer Diffraction and Light Scattering to measure the particle size and distribution in a dilute solution of particles. For small particle application to hair, the volume average particle diameters typically range from about 0.01 μm to about 4 μm, preferably from about 0.01 μm to about 2 μm, more preferably from about 0.01 μm to about 0.5 μm. For larger particle application to hair, the volume average particle diameters typically range from about 4 μm to about 50 μm, preferably from about 6 μm to about 40 μm, and more preferably from about 10 μm to about 35 μm.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

a. Silicone Oils

Silicone fluids include silicone oils, which are flowable silicone materials having a viscosity, as measured at 25° C., less than 1,000,000 csk, preferably from about 5 csk to about 1,000,000 csk, more preferably from about 100 csk to about 600,000 csk. Suitable silicone oils for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following Formula (III):

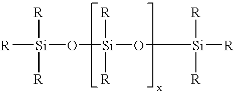

wherein R is aliphatic, preferably alkyl or alkenyl, or aryl, R can be substituted or unsubstituted, and x is an integer from 1 to about 8,000. Suitable R groups for use in the compositions of the present invention include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Preferred alkyl and alkenyl substituents are C₁ to C₅ alkyls and alkenyls, more preferably from C₁ to C₄, more preferably from C₁ to C₂. The aliphatic portions of other alkyl-, alkenyl-, or alkynyl-containing groups (such as alkoxy, alkaryl, and alkamino) can be straight or branched chains, and are preferably from C₁ to C₅, more preferably from C₁ to C₄, even more preferably from C₁ to C₃, more preferably from C₁ to C₂. As discussed above, the R substituents can also contain amino functionalities (e.g. alkamino groups), which can be primary, secondary or tertiary amines or quaternary ammonium. These include mono-, di- and tri-alkylamino and alkoxyamino groups, wherein the aliphatic portion chain length is preferably as described herein.

b. Amino and Cationic Silicones

Cationic silicone fluids suitable for use in the compositions of the present invention include, but are not limited to, those which conform to the general formula (V):

(R₁)ₐG₃₋ₐSi—(—OSiG₂)ₙ-(-SiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 0; b is 0 or 1, preferably 1; n is a number from 0 to 1,999, preferably from 49 to 499; m is an integer from 1 to 2,000, preferably from 1 to 10; the sum of n and m is a number from 1 to 2,000, preferably from 50 to 500; R₁ is a monovalent radical conforming to the general formula CqH_{2q}L, wherein q is an integer having a value from 2 to 8 and L is selected from the following groups:

—N(R₂)CH₂—CH₂—N(R₂)₂

—N(R₂)₂

—N(R₂)₃A⁻

—N(R₂)CH₂—CH₂—NR₂H₂A⁻

wherein R₂ is hydrogen, phenyl, benzyl, or a saturated hydrocarbon radical, preferably an alkyl radical from about C₁ to about C₂₀, and A⁻ is a halide ion.

An especially preferred cationic silicone corresponding to formula (V) is the polymer known as “trimethylsilylamodimethicone”, which is shown below in formula (VI):

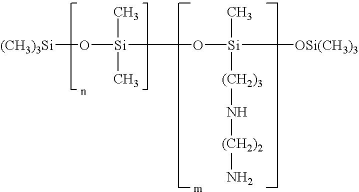

Other silicone cationic polymers which may be used in the compositions of the present invention are represented by the general formula (VII):

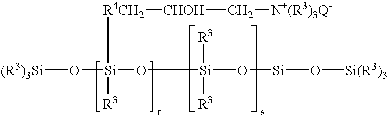

wherein R³ is a monovalent hydrocarbon radical from C₁ to C₁₈, preferably an alkyl or alkenyl radical, such as methyl; R⁴ is a hydrocarbon radical, preferably a C₁ to C₁₈ alkylene radical or a C₁₀ to C₁₈ alkyleneoxy radical, more preferably a C₁ to C₈ alkyleneoxy radical; Q⁻ is a halide ion, preferably chloride; r is an average statistical value from 2 to 20, preferably from 2 to 8; s is an average statistical value from 20 to 200, preferably from 20 to 50. A preferred polymer of this class is known as UCARE SILICONE ALE 56™, available from Union Carbide.

c. Silicone Gums

Other silicone fluids suitable for use in the compositions of the present invention are the insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76. Specific non-limiting examples of silicone gums for use in the compositions of the present invention include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

d. High Refractive Index Silicones

Other non-volatile, insoluble silicone fluid conditioning agents that are suitable for use in the compositions of the present invention are those known as “high refractive index silicones,” having a refractive index of at least about 1.46, preferably at least about 1.48, more preferably at least about 1.52, more preferably at least about 1.55. The refractive index of the polysiloxane fluid will generally be less than about 1.70, typically less than about 1.60. In this context, polysiloxane “fluid” includes oils as well as gums.

The high refractive index polysiloxane fluid includes those represented by general Formula (III) above, as well as cyclic polysiloxanes such as those represented by Formula (VIII) below:

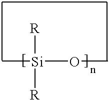

wherein R is as defined above, and n is a number from about 3 to about 7, preferably from about 3 to about 5.

The high refractive index polysiloxane fluids contain an amount of aryl-containing R substituents sufficient to increase the refractive index to the desired level, which is described herein. Additionally, R and n must be selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids will have a degree of aryl-containing substituents of at least about 15%, preferably at least about 20%, more preferably at least about 25%, even more preferably at least about 35%, more preferably at least about 50%. Typically, the degree of aryl substitution will be less than about 90%, more generally less than about 85%, preferably from about 55% to about 80%.

Preferred high refractive index polysiloxane fluids have a combination of phenyl or phenyl derivative substituents (more preferably phenyl), with alkyl substituents, preferably C₁-C₄ alkyl (more preferably methyl), hydroxy, or C₁-C₄ alkylamino (especially —R¹NHR²NH2 wherein each R¹ and R² independently is a C₁-C₃ alkyl, alkenyl, and/or alkoxy).

When high refractive index silicones are used in the compositions of the present invention, they are preferably used in solution with a spreading agent, such as a silicone resin or a surfactant, to reduce the surface tension by a sufficient amount to enhance spreading and thereby enhance the glossiness (subsequent to drying) of hair treated with the compositions.

Silicone fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

e. Silicone Resins

Silicone resins may be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

The weight ratio of the non-volatile silicone fluid, having refractive index below 1.46, to the silicone resin component, when used, is preferably from about 4:1 to about 400:1, more preferably from about 9:1 to about 200:1, more preferably from about 19:1 to about 100:1, particularly when the silicone fluid component is a polydimethylsiloxane fluid or a mixture of polydimethylsiloxane fluid and polydimethylsiloxane gum as described herein. Insofar as the silicone resin forms a part of the same phase in the compositions hereof as the silicone fluid, i.e. the conditioning active, the sum of the fluid and resin should be included in determining the level of silicone conditioning agent in the composition.

2. Organic Conditioning Oils

The conditioning component of the compositions of the present invention may also comprise from about 0.05% to about 3%, preferably from about 0.08% to about 1.5%, more preferably from about 0.1% to about 1%, of at least one organic conditioning oil as the conditioning agent, either alone or in combination with other conditioning agents, such as the silicones (described herein).

a. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C₁₂ to about C₁₉. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from Amoco Chemical Corporation. The concentration of such hydrocarbon oils in the composition preferably range from about 0.05% to about 20%, more preferably from about 0.08% to about 1.5%, and even more preferably from about 0.1% to about 1%.

b. Polyolefins

Organic conditioning oils for use in the compositions of the present invention can also include liquid polyolefins, more preferably liquid poly-α-olefins, more preferably hydrogenated liquid poly-α-olefins. Polyolefins for use herein are prepared by polymerization of C₄ to about C₁₄ olefenic monomers, preferably from about C₆ to about C₁₂.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Preferred hydrogenated α-olefin monomers include, but are not limited to: 1-hexene to 1-hexadecenes, 1-octene to 1-tetradecene, and mixtures thereof.

c. Fatty Esters

Other suitable organic conditioning oils for use as the conditioning agent in the compositions of the present invention include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

Specific examples of preferred fatty esters include, but are not limited to: iso-propyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are mono-carboxylic acid esters of the general formula R′COOR′, wherein R′ and R are alkyl or alkenyl radicals, and the sum of carbon atoms in R′ and R is at least 10, preferably at least 22.

Still other fatty esters suitable for use in the compositions of the present invention are di- and tri-alkyl and alkenyl esters of carboxylic acids, such as esters of C₄ to C₈ dicarboxylic acids (e.g. C₁ to C₂₂ esters, preferably C₁ to C₆, of succinic acid, glutaric acid, and adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the present invention are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other fatty esters suitable for use in the compositions of the present invention are glycerides, including, but not limited to, mono-, di-, and tri-glycerides, preferably di- and tri-glycerides, more preferably triglycerides. For use in the compositions described herein, the glycerides are preferably the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the compositions of the present invention are water insoluble synthetic fatty esters. Some preferred synthetic esters conform to the general Formula (IX):

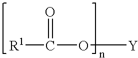

wherein R¹ is a C₇ to C₉ alkyl, alkenyl, hydroxyalkyl or hydroxyalkenyl group, preferably a saturated alkyl group, more preferably a saturated, linear, alkyl group; n is a positive integer having a value from 2 to 4, preferably 3; and Y is an alkyl, alkenyl, hydroxy or carboxy substituted alkyl or alkenyl, having from about 2 to about 20 carbon atoms, preferably from about 3 to about 14 carbon atoms. Other preferred synthetic esters conform to the general Formula (X):

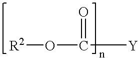

wherein R² is a C₈ to C₁₀ alkyl, alkenyl, hydroxyalkyl or hydroxyalkenyl group; preferably a saturated alkyl group, more preferably a saturated, linear, alkyl group; n and Y are as defined above in Formula (X).

Specific non-limiting examples of suitable synthetic fatty esters for use in the compositions of the present invention include: P-43 (C₈-C₁₀ triester of trimethylolpropane), MCP-684 (tetraester of 3,3 diethanol-1,5 pentadiol), MCP 121 (C₈-C₁₀ diester of adipic acid), all of which are available from Mobil Chemical Company.

3. Other Conditioning Agents

Also suitable for use in the compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586 (Clairol), 4,507,280 (Clairol), 4,663,158 (Clairol), 4,197,865 (L'Oreal), 4,217,914 (L'Oreal), 4,381,919 (L'Oreal), and 4,422,853 (L'Oreal).

4. Additional Components

The compositions of the present invention may further include a variety of additional useful components. Preferred additional components include those discussed below:

1. Other Anti-Microbial Actives

The compositions of the present invention may further include one or more anti-fungal or anti-microbial actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulfide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Preferred anti-microbials include itraconazole, ketoconazole, selenium sulphide and coal tar.

a. Azoles

Azole anti-microbials include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in the composition, the azole anti-microbial active is included in an amount from about 0.01% to about 5%, preferably from about 0.1% to about 3%, and more preferably from about 0.3% to about 2%, by weight of the composition. Especially preferred herein is ketoconazole.

b. Selenium Sulfide

Selenium sulfide is a particulate anti-dandruff agent suitable for use in the anti-microbial compositions of the present invention, effective concentrations of which range from about 0.1% to about 4%, by weight of the composition, preferably from about 0.3% to about 2.5%, more preferably from about 0.5% to about 1.5%. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 μm, as measured by forward laser light scattering device (e.g. Malvern 3600 instrument), preferably less than 10 μm. Selenium sulfide compounds are described, for example, in U.S. Pat. No. 2,694,668; U.S. Pat. No. 3,152,046; U.S. Pat. No. 4,089,945; and U.S. Pat. No. 4,885,107.

c. Sulfur

Sulfur may also be used as a particulate anti-microbial/anti-dandruff agent in the anti-microbial compositions of the present invention. Effective concentrations of the particulate sulfur are typically from about 1% to about 4%, by weight of the composition, preferably from about 2% to about 4%.

d. Keratolytic Agents

The present invention may further comprise one or more keratolytic agents such as Salicylic Acid.

Additional anti-microbial actives of the present invention may include extracts of melaleuca (tea tree) and charcoal. The present invention may also comprise combinations of anti-microbial actives. Such combinations may include octopirox and zinc pyrithione combinations, pine tar and sulfur combinations, salicylic acid and zinc pyrithione combinations, octopirox and climbasole combinations, and salicylic acid and octopirox combinations, and mixtures thereof.

2. Hair Loss Prevention and Hair Growth Agent

The present invention may further comprise materials useful for hair loss prevention and hair growth stimulants or agents. Examples of such agents are Anti-Androgens such as Propecia, Dutasteride, RU5884; Anti-Inflammatories such as Glucocortisoids, Macrolides, Macrolides; Anti-Microbials such as Zinc pyrithione, Ketoconazole, Acne Treatments; Immunosuppressives such as FK-506, Cyclosporin; Vasodilators such as minoxidil, Aminexil® and combinations thereof.

3. Sensates

The present invention may further comprise topical sensate materials such as terpenes, vanilloids, alkyl amides, natural extracts and combinations thereof, as demonstrated and disclosed in U.S. Application Ser. No. 11/216,520, filed Aug. 31, 2005 on page 39 and incorporated by reference herein.

4. Humectant

The compositions of the present invention may contain a humectant. The humectants herein are selected from the group consisting of polyhydric alcohols, water soluble alkoxylated nonionic polymers, and mixtures thereof as demonstrated and disclosed in U.S. application Ser. No. 11/216,520, filed Aug. 31, 2005 on page 39-40 and incorporated by reference herein.

5. Suspending Agent

The compositions of the present invention may further comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%. Suspending agents useful herein include anionic polymers and nonionic polymers, commercially available viscosity modifier, and crystalline suspending agents which can be categorized as acyl derivatives, long chain amine oxides, and mixtures thereof. as demonstrated and disclosed in U.S. application Ser. No. 11/216,520, filed Aug. 31, 2005 on page 40-42 and incorporated by reference herein.

6. Other Optional Components

The compositions of the present invention may contain also vitamins and amino acids, pigment material, antimicrobial agents which are useful as cosmetic biocides and antidandruff agents including: water soluble components such as piroctone olamine, water insoluble components such as 3,4,4′-trichlorocarbanilide (triclocarban), triclosan, chelating agents, as demonstrated and disclosed in U.S. application Ser. No. 11/216,520, filed Aug. 31, 2005, on page 42 and incorporated by reference herein.

The compositions of the present invention may also contain chelating agents.

### H. COORDINATING COMPOUND HAVING A LOG ZN BINDING CONSTANT

In an embodiment of the present invention, the composition further comprises a coordinating compound with a Log Zn binding constant in a range sufficient to maintain zinc bioavailability, as demonstrated and disclosed in U.S. application Ser. No. 11/216,520, filed Aug. 31, 2005 on pages 42-43 and incorporated by reference herein.

### I. pH

Preferably, the pH of the present invention may be greater than about 6.5, further wherein the pH is greater than about 6.8. Further, the pH of the present invention may be in a range from about 6.5 to about 12, preferably from about 6.8 to about 10, more preferably from about 6.8 to about 9, and even more preferably from about 6.8 to about 8.5.

### J. METHOD FOR ASSESSMENT OF ZINC LABILITY IN ZINC-CONTAINING PRODUCTS

Zinc lability is a measure of the chemical availability of zinc ion. Soluble zinc salts that do not complex with other species in solution have a relative zinc lability, by definition, of 100%. The use of partially soluble forms of zinc salts and/or incorporation in a matrix with potential complexants generally lowers the zinc lability substantially below the defined 100% maximum.

Zinc lability is assessed by combining a diluted zinc-containing solution or dispersion with the metallochromic dye xylenol orange (XO) and measurement of the degree of color change under specified conditions. The magnitude of color formation is proportional to the level of labile zinc. The procedure developed has been optimized for aqueous surfactant formulations but may be adapted to other physical product forms as well.

A spectrophotometer is used to quantify the color change at 572 nm, the wavelength of optimum color change for XO. The spectrophotometer is set to zero absorbance at 572 nm utilizing a product control as close in composition to the test product except excluding the potentially labile form of zinc. The control and test products are then treated identically as follows. A 50 μl product sample is dispensed into a jar and 95 ml of deaerated, distilled water are added and stirred. 5 mL of a 23 mg/mL xylenol orange stock solution at pH 5.0 is pipetted into the sample jar; this is considered time 0. The pH is then adjusted to 5.50±0.01 using dilute HCl or NaOH. After 10.0 minutes, a portion of the sample is filtered (0.45μ) and the absorbance measured at 572 nm. The measured absorbance is then compared to a separately measured control to determine the relative zinc lability (zero TO 100%). The 100% lability control is prepared in a matrix similar to the test products but utilizing a soluble zinc material (such as zinc sulfate) incorporated at an equivalent level on a zinc basis. The absorbance of the 100% lability control is measured as above for the test materials. The relative zinc lability is preferably greater than about 15%, more preferably greater than about 20%, and even more preferably greater than about 25%.

Using this methodology, the below examples demonstrate a material (basic zinc carbonate) that has intrinsically high lability in an anionic surfactant system compared to one (ZnO) with low intrinsic lability.

| | | Relative Zinc | |
|---|---|---|---|
| | Relative Zinc | Lability (%) | |
| | Lability (%) | In Simple Surfactant | |
| | In Water | System¹ | Lability Benefit |
| Zinc Oxide | 86.3 | 1.5 | NO |
| Basic zinc | 100 | 37 | YES |
| carbonate | | | |

¹Simple surfactant system: 6% sodium lauryl sulfate

### K. PARTICLE SIZE DETERMINATION METHOD

Particle size analyses on zinc oxide and hydrozincite raw materials as demonstrated and disclosed in U.S. application Ser. No. 11/216,520, filed Aug. 31, 2005 on pages 44-45 are incorporated by reference herein.

### L. SURFACE AREA METHODOLOGY

Surface area analysis as demonstrated and disclosed in U.S. application Ser. No. 11/216,520, filed Aug. 31, 2005 on page 45 are incorporated by reference herein.

### M. METHODS OF USE

The compositions of the present invention may be used in direct application to the skin or in a conventional manner for cleansing skin and hair and controlling microbial infection (including fungal, viral, or bacterial infections) on the skin or scalp. The compositions herein are useful for cleansing the hair and scalp, and other areas of the body such as underarm, feet, and groin areas and for any other area of skin in need of treatment. The present invention may be used for treating or cleansing of the skin or hair of animals as well. An effective amount of the composition, typically from about 1 g to about 50 g, preferably from about 1 g to about 20 g of the composition, for cleansing hair, skin or other area of the body, is topically applied to the hair, skin or other area that has preferably been wetted, generally with water, and then rinsed off. Application to the hair typically includes working the shampoo composition through the hair.

A further embodiment of the present invention comprises a method of treating athlete's foot, microbial infections, improving the appearance of a scalp, fungal infections, diaper dermatitis, tinea capitis, yeast infections and candidiasis, each comprising the use of the composition according to the present invention.

Additional methods for providing anti-microbial (i.e. anti-dandruff) efficacy as demonstrated and disclosed in U.S. application Ser. No. 11/216,520, filed Aug. 31, 2005 on pages 46-47 are incorporated by reference herein.

### N. EXAMPLES

The following examples further describe and demonstrate the preferred embodiments within the scope of the present invention. The examples are given solely for the purpose of illustration, and are not to be construed as limitations of the present invention since many variations thereof are possible without departing from its scope.

The composition of the invention can be made by mixing one or more selected metal ion sources and one or more metal salts of pyrithione in an appropriate media or carrier, or by adding the individual components separately to the skin or hair cleansing compositions. Useful carriers are discussed more fully above.

1. Topical Compositions

All exemplified compositions can be prepared by conventional formulation and mixing techniques. Component amounts are listed as weight percents and exclude minor materials such as diluents, filler, and so forth. The listed formulations, therefore, comprise the listed components and any minor materials associated with such components. As used herein, “minors” refers to those optional components such as preservatives, viscosity modifiers, pH modifiers, fragrances, foam boosters, and the like. As is apparent to one of ordinary skill in the art, the selection of these minors will vary depending on the physical and chemical characteristics of the particular ingredients selected to make the present invention as described herein. Other modifications can be undertaken by the skilled artisan without departing from the spirit and scope of this invention.

### O. METHODS OF MANUFACTURE FOR SHAMPOO COMPOSITIONS

Methods of Manufacture for Shampoo Compositions

The compositions of the present invention may be prepared by any known or otherwise effective technique, suitable for providing an anti-microbial composition provided that the resulting composition provides the excellent anti-microbial benefits described herein. Methods for preparing the anti-dandruff and conditioning shampoo embodiments of the present invention include conventional formulation and mixing techniques. A method such as that described in U.S. Pat. No. 5,837,661, could be employed, wherein the anti-microbial agent of the present invention would typically be added in the same step as the silicone premix is added in the U.S. Pat. No. 5,837,661 description.

### Antimicrobial Shampoo—Examples

A suitable method for preparing the anti-microbial shampoo compositions described in Examples 1 to 26 (below) follows:

About one-third to all of the sodium laureth sulfate (added as 29 wt % solution) and acid are added to a jacketed mix tank and heated to about 60° C. to about 80° C. with slow agitation to form a surfactant solution. The pH of this solution is about 3 to about 7. Sodium benzoate, Cocoamide MEA and fatty alcohols, (where applicable), are added to the tank and allowed to disperse. Ethylene glycol distearate (“EGDS”) is added to the mixing vessel and allowed to melt (where applicable). After the EGDS is melted and dispersed, Kathon CG is added to the surfactant solution. The resulting mixture is cooled to about 25° C. to about 40° C. and collected in a finishing tank. As a result of this cooling step, the EGDS crystallizes to form a crystalline network in the product (where applicable). The remainder of the sodium laureth sulfate and other components, including the silicone and anti-microbial agent(s), are added to the finishing tank with agitation to ensure a homogeneous mixture. Polymers (cationic or nonionic) are dispersed in water or oils as an about 0.1% to about 10% dispersion and/or solution, or, alternatively, added as is, and can be added to the main mix, final mix, or both. Basic Zinc Carbonate or other zinc-containing layered material can be added to a premix of surfactants or water with or without the aid of a dispersing agent via conventional powder incorporation and mixing techniques into the final mix. Once all components have been added, additional viscosity modifiers, such as sodium chloride and/or sodium xylenesulfonate may be added, as needed, to adjust product viscosity to the extent desired. Product pH can be adjusted, using an acid such as hydrochloric acid, to an acceptable value.

### Non-Limiting Examples

| | EXAMPLE | | | | | | |
|---|---|---|---|---|---|---|---|
| COMPOSITION | 1 | 2 | 3 | 4 | 5 | 6 | 7 |
| Sodium Laureth Sulfate | | 10.0 | 6.50 | 6.50 | 6.50 | 7.5 | 7.5 |
| (SE₃S) | | | | | | | |
| Sodium Lauryl Sulfate (SLS) | 2.0 | 6.0 | 5.50 | 5.50 | 5.50 | 6.5 | 6.5 |
| Decyl glucoside | 10.0 | | | | | | |
| Sodium Lauroamphoacetate⁽³²⁾ | | | | | 2.00 | | |
| Laureth-4⁽³³⁾ | | | 0.90 | 0.90 | | | |
| Cocamidopropyl Betaine | 2.0 | | | 1.00 | | | |
| Cocamide MEA | 0.80 | 0.80 | 0.80 | 0.80 | 0.80 | | 0.80 |
| Cetyl Alcohol | 0.60 | 0.60 | 0.60 | 0.60 | 0.60 | | 0.60 |
| Dihydrogenated | | | 0.15 | 0.15 | 0.15 | | |
| Tallowamidoethyl | | | | | | | |
| Hydroxyethylmonium | | | | | | | |
| Methosulfate⁽³⁴⁾ | | | | | | | |
| 1-Propanaminium, N,N,N- | 0.40⁽¹⁾ | 0.50⁽²⁾ | | | | | |
| trimethyl-3-[(2-methyl-1-oxo- | | | | | | | |
| 2-propenyl)amino]-, | | | | | | | |
| chloride; | | | | | | | |
| (Poly(Methacrylamidopropyl | | | | | | | |
| trimethyl ammonium | | | | | | | |
| chloride))^{(1,2)} | | | | | | | |
| Methacryloamidopropyl- | | | 0.75 | | | | |
| pentamethyl-1,3-propylene- | | | | | | | |
| 2-ol-ammonium dichloride⁽³⁾ | | | | | | | |
| N,N,N,N′,N′,N″,N″- | | | | | | | 0.40 |
| heptamethyl-N″-3-(1-oxo-2- | | | | | | | |
| methyl-2- | | | | | | | |
| propenyl)aminopropyl-9- | | | | | | | |
| oxo-8-azo-decane-1,4,10- | | | | | | | |
| triammonium trichloride⁽¹²⁾ | | | | | | | |
| 1-Propanaminium, N,N,N- | | | | 0.50 | | | |
| trimethyl-3-[(1-oxo-2- | | | | | | | |
| propenyl)amino]-, chloride; | | | | | | | |
| (Poly(Acrylamidopropyl | | | | | | | |
| trimethyl ammonium | | | | | | | |
| chloride))⁽⁴⁾ | | | | | | | |
| [3- | | | | | 0.75 | | |
| methacryloylamino)propyl] | | | | | | | |
| dimethylethylammonium | | | | | | | |
| ethylsulfate homopolymer⁽⁵⁾ | | | | | | | |
| [(2- | | | | | | 1.50 | |
| methacryloyloxy)ethyl]trimethylammonium | | | | | | | |
| methylsulfate | | | | | | | |
| homopolymer⁽⁶⁾ | | | | | | | |
| Trimethylammoniopropyl | | | | | | | 0.50 |
| methacyryl- | | | | | | | |
| Amide chloride-N- | | | | | | | |
| Hydroxyethyl acrylate | | | | | | | |
| copolymer⁽⁷⁾ | | | | | | | |
| Trimethylammoniopropyl | | | | | | | |
| methacyryl- | | | | | | | |
| Amide chloride-N- | | | | | | | |
| vinylpyrrolidone copolymer⁽⁸⁾ | | | | | | | |
| Dimethyldiallyl ammonium | | | | | | | |
| chloride-N-b- | | | | | | | |
| hydroxyethylacrylate | | | | | | | |
| copolymer⁽⁹⁾ | | | | | | | |
| Trimethylammoniopropyl | | | | | | | |
| methacyryl- | | | | | | | |
| Amide chloride-N- | | | | | | | |
| Methacrylamido- | | | | | | | |
| propyldimethyl-ammonium | | | | | | | |
| methyl-carboxylate | | | | | | | |
| copolymer⁽¹⁰⁾ | | | | | | | |
| 2-Propen-1-aminium, N,N- | | | | | | | |
| Dimethyl-N-2-Propenyl-, | | | | | | | |
| Chloride Homopolymer⁽¹¹⁾ | | | | | | | |
| Guar | | | | | | | |
| hydroxypropyltrimonium | | | | | | | |
| chloride⁽¹³⁾ | | | | | | | |
| Polyquaternium-10⁽¹⁴⁾ | | | | | | | |
| Cassia polymer⁽¹⁵⁾ | | | | | | | |
| Ethylene Glycol Distearate | | 1.50 | 1.50 | 1.50 | 1.50 | 1.50 | 3.0 |
| Trihydroxystearin⁽¹⁶⁾ | 0.25 | | | | | | |
| Polyethylene Glycol (14000)⁽¹⁷⁾ | | | 0.17 | 0.17 | 0.17 | 0.17 | 0.17 |
| Zinc pyrithione⁽¹⁸⁾ | 1.0 | 1.0 | 1.0 | 1.0 | 0.50 | 1.0 | 4.0 |
| Basic zinc carbonate⁽¹⁹⁾ | | | | | 0.80 | 1.61 | 3.2 |
| Zinc hydroxysulfate⁽²⁰⁾ | 2.0 | | | | | | |
| Zinc hydroxynitrate⁽²⁰⁾ | | 1.88 | | | | | |
| Zinc hydroxychloride⁽²⁰⁾ | | | 1.63 | | | | |
| Zinc hydroxylauryl sulfate⁽²⁰⁾ | | | | 2.40 | | | |
| Magnesium sulfate | 0.28 | 0.28 | 0.28 | 0.28 | 0.28 | 0.28 | 0.28 |
| Sodium Benzoate | 0.25 | 0.25 | 0.25 | 0.25 | 0.25 | 0.25 | 0.25 |
| Benzyl alcohol | .0225 | .0225 | .0225 | .0225 | .0225 | .0225 | .0225 |
| 5-chloro-2-methyl-4- | .0006 | .0006 | .0006 | .0006 | .0006 | .0006 | .0006 |
| isothiazolin-3-one | | | | | | | |
| Dimethicone^{(21,22,23)} | 1.0⁽²¹⁾ | 2.35⁽²¹⁾ | 0.50⁽²¹⁾ | 2.00⁽²²⁾ | 2.00⁽²³⁾ | 0.85⁽²¹⁾ | 0.85⁽²¹⁾ |
| Polydecene⁽²⁴⁾ | | 0.40 | | | | | |
| Trimethylolpropane | | 0.10 | | | | | |
| Tricaprylate/Tricaprate⁽²⁵⁾ | | | | | | | |
| Silica^{(26,27,28,29)} | | 1.25⁽²⁶⁾ | 2.25⁽²⁷⁾ | 0.50⁽²⁸⁾ | 0.25⁽²⁹⁾ | | |
| Polymethylsilsesquioxane^{(30,31)} | 1.20⁽³⁰⁾ | | | | | 3.0⁽³⁰⁾ | 5.0⁽³¹⁾ |
| Fragrance | 0.4 | 0.75 | 1.25 | 0.4 | 0.75 | 1.25 | 0.4 |
| Sodium chloride/sodium | Visc | Visc | Visc | Visc | Visc | Visc | Visc |
| xylene sulfonate | QS | QS | QS | QS | QS | QS | QS |
| Hydrochloric acid | pH | pH | pH | pH | pH | pH | pH |
| | QS | QS | QS | QS | QS | QS | QS |
| Water and Minors (QS to | | | | | | | |
| 100%) | | | | | | | |

| | EXAMPLE | | | | | |
|---|---|---|---|---|---|---|
| COMPOSITION | 8 | 9 | 10 | 11 | 12 | 13 |
| Sodium Laureth Sulfate | 6.50 | 6.50 | 7.50 | 10.0 | 10.0 | 10.0 |
| (SE₃S) | | | | | | |
| Sodium Lauryl Sulfate (SLS) | 5.50 | 5.50 | 6.50 | 6.0 | 6.0 | 6.0 |
| Decyl glucoside | | | | | | |
| Sodium Lauroamphoacetate⁽³²⁾ | | 2.00 | | | | |
| Laureth-4⁽³³⁾ | | | | | | |
| Cocamidopropyl Betaine | | | | | | |
| Cocamide MEA | 0.80 | 0.80 | | 1.60 | 1.60 | 1.60 |
| Cetyl Alcohol | 0.60 | 0.60 | | 0.60 | 0.60 | 0.60 |
| Dihydrogenated | 0.15 | 0.15 | | | | |
| Tallowamidoethyl | | | | | | |
| Hydroxyethylmonium | | | | | | |
| Methosulfate⁽³⁴⁾ | | | | | | |
| 1-Propanaminium, N,N,N- | | | | | | |
| trimethyl-3-[(2-methyl-1-oxo- | | | | | | |
| 2-propenyl)amino]-, | | | | | | |
| chloride; | | | | | | |
| (Poly(Methacrylamidopropyl | | | | | | |
| trimethyl ammonium | | | | | | |
| chloride))^{(1,2)} | | | | | | |
| Methacryloamidopropyl- | | | | | | |
| pentamethyl-1,3-propylene- | | | | | | |
| 2-ol-ammonium dichloride⁽³⁾ | | | | | | |
| N,N,N,N′,N′,N″,N″- | | | | | | |
| heptamethyl-N″-3-(1-oxo-2- | | | | | | |
| methyl-2- | | | | | | |
| propenyl)aminopropyl-9- | | | | | | |
| oxo-8-azo-decane-1,4,10- | | | | | | |
| triammonium trichloride⁽¹²⁾ | | | | | | |
| 1-Propanaminium, N,N,N- | | | | | | |
| trimethyl-3-[(1-oxo-2- | | | | | | |
| propenyl)amino]-, chloride; | | | | | | |
| (Poly(Acrylamidopropyl | | | | | | |
| trimethyl ammonium | | | | | | |
| chloride))⁽⁴⁾ | | | | | | |
| [3- | | | | | | |
| methacryloylamino)propyl] | | | | | | |
| dimethylethylammonium | | | | | | |
| ethylsulfate homopolymer⁽⁵⁾ | | | | | | |
| [(2- | | | | | | |
| methacryloyloxy)ethyl]trimethylammonium | | | | | | |
| methylsulfate | | | | | | |
| homopolymer⁽⁶⁾ | | | | | | |
| Trimethylammoniopropyl | | | | | | |
| methacyryl- | | | | | | |
| Amide chloride-N- | | | | | | |
| Hydroxyethyl acrylate | | | | | | |
| copolymer⁽⁷⁾ | | | | | | |
| Trimethylammoniopropyl | 2.0 | | | | | |
| methacyryl- | | | | | | |
| Amide chloride-N- | | | | | | |
| vinylpyrrolidone copolymer⁽⁸⁾ | | | | | | |
| Dimethyldiallyl ammonium | | 5.0 | | | | |
| chloride-N-b- | | | | | | |
| hydroxyethylacrylate | | | | | | |
| copolymer⁽⁹⁾ | | | | | | |
| Trimethylammoniopropyl | | | 5.0 | | | |
| methacyryl- | | | | | | |
| Amide chloride-N- | | | | | | |
| Methacrylamido- | | | | | | |
| propyldimethyl-ammonium | | | | | | |
| methyl-carboxylate | | | | | | |
| copolymer⁽¹⁰⁾ | | | | | | |
| 2-Propen-1-aminium, N,N- | | | | 2.5 | 0.25 | 0.025 |
| Dimethyl-N-2-Propenyl-, | | | | | | |
| Chloride Homopolymer⁽¹¹⁾ | | | | | | |
| Guar | | | | | | |
| hydroxypropyltrimonium | | | | | | |
| chloride⁽¹³⁾ | | | | | | |
| Polyquaternium-10⁽¹⁴⁾ | | | | | | |
| Cassia polymer⁽¹⁵⁾ | | | | | | |
| Ethylene Glycol Distearate | 2.50 | 1.50 | 1.50 | 1.50 | | 1.50 |
| Trihydroxystearin⁽¹⁶⁾ | | | | | 0.25 | |
| Polyethylene Glycol (14000)⁽¹⁷⁾ | 0.17 | | 0.17 | | | |
| Zinc pyrithione⁽¹⁸⁾ | 2.0 | .8 | 1.0 | 1.0 | 1.0 | 1.0 |
| Basic zinc carbonate⁽¹⁹⁾ | 1.61 | 1.0 | 0.50 | 1.61 | 1.61 | 1.61 |
| Zinc hydroxysulfate⁽²⁰⁾ | | | | | | |
| Zinc hydroxynitrate⁽²⁰⁾ | | | | | | |
| Zinc hydroxychloride⁽²⁰⁾ | | | | | | |
| Zinc hydroxylauryl sulfate⁽²⁰⁾ | | | | | | |
| Magnesium sulfate | 0.28 | 0.28 | 0.28 | 0.28 | 0.28 | 0.28 |
| Sodium Benzoate | 0.25 | 0.25 | 0.25 | 0.25 | 0.25 | 0.25 |
| Benzyl alcohol | .0225 | .0225 | .0225 | .0225 | .0225 | .0225 |
| 5-chloro-2-methyl-4- | .0006 | .0006 | .0006 | .0006 | .0006 | .0006 |
| isothiazolin-3-one | | | | | | |
| Dimethicone^{(21,22,23)} | 0.50⁽²¹⁾ | 1.0⁽²¹⁾ | 0.25⁽²¹⁾ | 1.0⁽²¹⁾ | 1.0⁽²¹⁾ | 0.50⁽²¹⁾ |
| Polydecene⁽²⁴⁾ | | | | | | |
| Trimethylolpropane | | | | | | |
| Tricaprylate/Tricaprate⁽²⁵⁾ | | | | | | |
| Silica^{(26,27,28,29)} | | | | | | |
| Polymethylsilsesquioxane^{(30,31)} | | | | 0.25⁽³¹⁾ | | |
| Fragrance | 0.75 | 1.25 | 0.4 | 0.75 | 0.75 | 0.75 |
| Sodium chloride/sodium | Visc | Visc | Visc | Visc | Visc | Visc |
| xylene sulfonate | QS | QS | QS | QS | QS | QS |
| Hydrochloric acid | pH | pH | pH | pH | pH | pH |
| | QS | QS | QS | QS | QS | QS |
| Water and Minors (QS to | | | | | | |
| 100%) | | | | | | |

⁽¹⁾HMW MAPTAC (Rhodia) [charge density = 4.5 meq/g, molecular weight ~860,000]
⁽²⁾HHMW MAPTAC (Rhodia) [charge density = 4.5 meq/g, molecular weight ~1,500,000]
⁽³⁾Diquat (Rhodia) [charge density = 5.60 meq/g, molecular weight ~252,000]
⁽⁴⁾APTAC (Rhodia) [charge density = 4.88 meq/g, molecular weight ~1,916,000]
⁽⁵⁾Homopolymer of DMAPMA + DES (Rhodia) [charge density = 3.09 meq/g, molecular weight ~180,000]
⁽⁶⁾Homopolymer of METAMS (Rhodia) [charge density = 3.53 meq/g, molecular weight ~313,000]
⁽⁷⁾any one of the following: 1:9 HEA:MAPTAC (Rhodia) [cationic density = 4.29 meq/g, MW ~276.000], 3:7HEA:MAPTAC (Rhodia) [cationic density = 3.71 meq/g, MW ~648,000], 3:7HEA:MAPTAC (Rhodia)
[cationic density = 3.71 meq/g, MW ~1,200,000]
⁽⁸⁾any one of the following: 1:9 VP:MAPTAC (Rhodia) [cationic density = 4.30 meq/g. MW~242,000], or 3:7 VP:MAPTAC (Rhodia) [cationic density = 3.74 meq/g. MW~503,000]
⁽⁹⁾1:9 HEA:DMDAAC (Rhodia) [cationic density = 5.75 meq/g, MW ~274.000]
⁽¹⁰⁾1:1 AP:MAPTAC (Rhodia) [cationic density = 3.95 meq/g, MW ~243,000]
⁽¹¹⁾Polyquaternium 6 (Mirapol 100, available from Rhodia)
⁽¹²⁾Triquat (Rhodia, charge density = 6.07 meq/g)
⁽¹³⁾NHance 3269 (available from Aqualon/Hercules)
⁽¹⁴⁾Polymer LR400 (available from Amerchol/Dow)
⁽¹⁵⁾Cassia galactomannan (MW ~200,000, charge density = 0.7 or 3.0 meq/g)
⁽¹⁶⁾Thixcin R (Rheox)
⁽¹⁷⁾PEG 14M (Dow Chemical)
⁽¹⁸⁾ZPT (having an average particle size of about 2.5 micron, available from Arch)
⁽¹⁹⁾available from Bruggemann Chemical
⁽²⁰⁾Materials made by reported methods in Lagaly, G: et al. Inorg. Chem. 3, 32, 1209-1215 & Morioka, H; et al. Inorg chem.. 1999, 38, 4211-4216.
⁽²¹⁾Viscasil 330M (General Electric Silicones)
⁽²²⁾Dow Corning ® 1664 Emulsion (Dow Corning)
⁽²³⁾Dow Corning ® 2-1865 Microemulsion (Dow Corning)
⁽²⁴⁾Puresyn 6, MCP-1812 (Mobil)
⁽²⁵⁾Mobil P43 (Mobil)
⁽²⁶⁾Sipernat 22LS (Degussa)
⁽²⁷⁾MSS-500H (GE Silicones)
⁽²⁸⁾MSS-500N (GE Silicones)
⁽²⁹⁾Syloid 244FP Silica (Grace Davison)
⁽³⁰⁾Tospearl 240 (GE Silicones)
⁽³¹⁾Tospearl 3120 (GE Silicones)
⁽³²⁾Miranol Ultra L32 (Rhodia)
⁽³³⁾Brij 30
⁽³⁴⁾Varisoft 110 (Witco)

| | EXAMPLE | | | | | | | | | | | | |
|---|---|---|---|---|---|---|---|---|---|---|---|---|---|
| COMPOSITION | 14 | 15 | 16 | 17 | 18 | 19 | 20 | 21 | 22 | 23 | 24 | 25 | 26 |
| Sodium Laureth Sulfate | 6.0 | 6.0 | 6.0 | 6.0 | 6.0 | 6.0 | 10.0 | 10.0 | 10.0 | 7.65 | 10.0 | 10.0 | 10.0 |
| (SE₃S) | | | | | | | | | | | | | |
| Sodium Lauryl Sulfate | 10.0 | 10.0 | 10.0 | 7.0 | 7.0 | 7.0 | 6.0 | 6.0 | 6.0 | 6.35 | 6.0 | 6.0 | 6.0 |
| (SLS) | | | | | | | | | | | | | |
| Laureth-4⁽³³⁾ | 0.90 | 0.90 | 0.90 | | | | | | | | | | |
| Cocamidopropyl Betaine | | | | 2.0 | 2.0 | 2.0 | | | | | | | |
| Cocamide MEA | | | | 0.85 | 0.85 | 0.85 | 1.60 | 1.60 | 1.60 | | 1.60 | 1.60 | 1.60 |
| Cetyl Alcohol | | | | | | | 0.6 | 0.6 | 0.6 | | 0.60 | 0.60 | 0.60 |
| Dihydrogenated | | | | | | | | | | | | | |
| Tallowamidoethyl | | | | | | | | | | | | | |
| Hydroxyethylmonium | | | | | | | | | | | | | |
| Methosulfate⁽³⁴⁾ | | | | | | | | | | | | | |
| 1-Propanaminium, | | | | | | | | | | | | | |
| N,N,N-trimethyl-3-[(2- | | | | | | | | | | | | | |
| methyl-1-oxo-2- | | | | | | | | | | | | | |
| propenyl)amino]-, | | | | | | | | | | | | | |
| chloride; | | | | | | | | | | | | | |
| (Poly- | | | | | | | | | | | | | |
| (Methacrylamidopropyl | | | | | | | | | | | | | |
| trimethyl | | | | | | | | | | | | | |
| ammonium chloride))^{(1,2)} | | | | | | | | | | | | | |
| Methacryloamidopropyl- | | | | | | | | | | | | | |
| pentamethyl-1,3- | | | | | | | | | | | | | |
| propylene-2-ol- | | | | | | | | | | | | | |
| ammonium dichloride⁽³⁾ | | | | | | | | | | | | | |
| N,N,N,N′,N′,N″,N″- | | | | | | | | | | | | | |
| heptamethyl-N″-3-(1- | | | | | | | | | | | | | |
| oxo-2-methyl-2- | | | | | | | | | | | | | |
| propenyl)aminopropyl-9- | | | | | | | | | | | | | |
| oxo-8-azo-decane- | | | | | | | | | | | | | |
| 1,4,10-triammonium | | | | | | | | | | | | | |
| trichloride⁽¹²⁾ | | | | | | | | | | | | | |
| 1-Propanaminium, | | | | | | | | | | | | | |
| N,N,N-trimethyl-3-[(1- | | | | | | | | | | | | | |
| oxo-2-propenyl)amino]-, | | | | | | | | | | | | | |
| chloride; | | | | | | | | | | | | | |
| (Poly(Acrylamidopropyl | | | | | | | | | | | | | |
| trimethyl ammonium | | | | | | | | | | | | | |
| chloride))⁽⁴⁾ | | | | | | | | | | | | | |
| [3- | | | | | | | | | | | | | |
| methacryloylamino)- | | | | | | | | | | | | | |
| propyl] | | | | | | | | | | | | | |
| dimethylethylammonium | | | | | | | | | | | | | |
| ethylsulfate | | | | | | | | | | | | | |
| homopolymer⁽⁵⁾ | | | | | | | | | | | | | |
| [(2- | | | | | | | | | | | | | |
| methacryloyloxy)ethyl]tri | | | | | | | | | | | | | |
| methylammonium | | | | | | | | | | | | | |
| methylsulfate | | | | | | | | | | | | | |
| homopolymer⁽⁶⁾ | | | | | | | | | | | | | |
| Trimethylammoniopropyl | | | | | | | | | | | | | |
| methacyryl- | | | | | | | | | | | | | |
| Amide chloride-N- | | | | | | | | | | | | | |
| Hydroxyethyl acrylate | | | | | | | | | | | | | |
| copolymer⁽⁷⁾ | | | | | | | | | | | | | |
| Trimethylammoniopropyl | | | | | | | | | | | | | |
| methacyryl- | | | | | | | | | | | | | |
| Amide chloride-N- | | | | | | | | | | | | | |
| vinylpyrrolidone | | | | | | | | | | | | | |
| copolymer⁽⁸⁾ | | | | | | | | | | | | | |
| Dimethyldiallyl | | | | | | | | | | | | | |
| ammonium chloride-N-b- | | | | | | | | | | | | | |
| hydroxyethylacrylate | | | | | | | | | | | | | |
| copolymer⁽⁹⁾ | | | | | | | | | | | | | |
| Trimethylammoniopropyl | | | | | | | | | | | | | |
| methacyryl- | | | | | | | | | | | | | |
| Amide chloride-N- | | | | | | | | | | | | | |
| Methacrylamido- | | | | | | | | | | | | | |
| propyldimethyl- | | | | | | | | | | | | | |
| ammonium methyl- | | | | | | | | | | | | | |
| carboxylate | | | | | | | | | | | | | |
| copolymer⁽¹⁰⁾ | | | | | | | | | | | | | |
| 2-Propen-1-aminium, | 0.25 | 0.10 | 0.05 | 0.25 | 0.10 | 0.05 | 0.25 | 0.10 | 0.05 | 0.10 | 0.025 | 0.025 | 0.25 |
| N,N-Dimethyl-N-2- | | | | | | | | | | | | | |
| Propenyl-, Chloride | | | | | | | | | | | | | |
| Homopolymer⁽¹¹⁾ | | | | | | | | | | | | | |
| Guar | | | | | | | | | | | 0.25 | | |
| hydroxypropyltrimonium | | | | | | | | | | | | | |
| chloride⁽¹³⁾ | | | | | | | | | | | | | |
| Polyquaternium-10⁽¹⁴⁾ | | | | | | | | | | | | 0.25 | |
| Cassia polymer⁽¹⁵⁾ | | | | | | | | | | | | | 0.25 |
| Ethylene Glycol | 1.50 | 1.50 | 1.50 | 1.50 | 1.50 | 1.50 | 1.50 | 1.50 | 1.50 | 1.50 | 1.50 | 1.50 | 1.50 |
| Distearate | | | | | | | | | | | | | |
| Trihydroxystearin⁽¹⁶⁾ | | | | | | | | | | | | | |
| Polyethylene Glycol | | | | | | | | | | | | | |
| (14000)⁽¹⁷⁾ | | | | | | | | | | | | | |
| Zinc pyrithione⁽¹⁸⁾ | 1.0 | 1.0 | 1.0 | 1.0 | 1.0 | 1.0 | 1.0 | 1.0 | 1.0 | 1.0 | 1.0 | 1.0 | 1.0 |
| Basic zinc carbonate⁽¹⁹⁾ | 1.61 | 1.61 | 1.61 | 1.61 | 1.61 | 1.61 | 1.61 | 1.61 | 1.61 | 1.61 | 1.61 | 1.61 | 1.61 |
| Zinc hydroxysulfate⁽²⁰⁾ | | | | | | | | | | | | | |
| Zinc hydroxynitrate⁽²⁰⁾ | | | | | | | | | | | | | |
| Zinc hydroxychloride⁽²⁰⁾ | | | | | | | | | | | | | |
| Zinc hydroxylauryl | | | | | | | | | | | | | |
| sulfate⁽²⁰⁾ | | | | | | | | | | | | | |
| Magnesium sulfate | 0.28 | 0.28 | 0.28 | 0.28 | 0.28 | 0.28 | 0.28 | 0.28 | 0.28 | 0.28 | 0.28 | 0.28 | 0.28 |
| Sodium Benzoate | 0.25 | 0.25 | 0.25 | 0.25 | 0.25 | 0.25 | 0.25 | 0.25 | 0.25 | 0.25 | 0.25 | 0.25 | 0.25 |
| Benzyl alcohol | .0225 | .0225 | .0225 | .0225 | .0225 | .0225 | .0225 | .0225 | .0225 | .0225 | .0225 | .0225 | .0225 |
| 5-chloro-2-methyl-4- | .0006 | .0006 | .0006 | .0006 | .0006 | .0006 | .0006 | .0006 | .0006 | .0006 | .0006 | .0006 | .0006 |
| isothiazolin-3-one | | | | | | | | | | | | | |
| Dimethicone^{(21,22,23)} | 2.0⁽²¹⁾ | 1.0⁽²¹⁾ | 0.50⁽²¹⁾ | 2.0⁽²¹⁾ | 1.0⁽²¹⁾ | 0.50⁽²¹⁾ | 2.0⁽²¹⁾ | 1.0⁽²¹⁾ | 0.50⁽²¹⁾ | | 2.0⁽²¹⁾ | 2.0⁽²¹⁾ | 2.0⁽²²⁾ |
| Polydecene⁽²⁴⁾ | | | | | | | | | | | | | |
| Trimethylolpropane | | | | | | | | | | | | | |
| Tricaprylate/ | | | | | | | | | | | | | |
| Tricaprate⁽²⁵⁾ | | | | | | | | | | | | | |
| Fragrance | 0.75 | 0.75 | 0.75 | 0.75 | 0.75 | 0.75 | 0.75 | 0.75 | 0.75 | 0.75 | 0.75 | 0.75 | 0.75 |
| Sodium chloride/sodium | Visc | Visc | Visc | Visc | Visc | Visc | Visc | Visc | Visc | Visc | Visc | Visc | Visc |
| xylene sulfonate | QS | QS | QS | QS | QS | QS | QS | QS | QS | QS | QS | QS | QS |
| Hydrochloric acid | pH | pH | pH | pH | pH | pH | pH | pH | pH | pH | pH | pH | pH |
| | QS | QS | QS | QS | QS | QS | QS | QS | QS | QS | QS | QS | QS |
| Water and Minors (QS | | | | | | | | | | | | | |
| to 100%) | | | | | | | | | | | | | |

⁽¹⁾HMW MAPTAC (Rhodia) [charge density = 4.5 meq/g, molecular weight ~860,000]
⁽²⁾HHMW MAPTAC (Rhodia) [charge density = 4.5 meq/g, molecular weight ~1,500,000]
⁽³⁾Diquat (Rhodia) [charge density = 5.60 meq/g, molecular weight ~252,000]
⁽⁴⁾APTAC (Rhodia) [charge density = 4.88 meq/g, molecular weight ~1,916,000]
⁽⁵⁾Homopolymer of DMAPMA + DES (Rhodia) [charge density = 3.09 meq/g, molecular weight ~180,000]
⁽⁶⁾Homopolymer of METAMS (Rhodia) [charge density = 3.53 meq/g, molecular weight ~313,000]
⁽⁷⁾any one of the following: 1:9 HEA:MAPTAC (Rhodia) [cationic density = 4.29 meq/g, MW ~276.000], 3:7HEA:MAPTAC (Rhodia) [cationic density = 3.71 meq/g, MW ~648,000], 3:7HEA:MAPTAC (Rhodia) [cationic density = 3.71 meq/g, MW ~1,200,000]
⁽⁸⁾any one of the following: 1:9 VP:MAPTAC (Rhodia) [cationic density = 4.30 meq/g. MW~242,000], or 3:7 VP:MAPTAC (Rhodia) [cationic density = 3.74 meq/g. MW~503,000]
⁽⁹⁾1:9 HEA:DMDAAC (Rhodia) [cationic density = 5.75 meq/g, MW ~274.000]
⁽¹⁰⁾1:1 AP:MAPTAC (Rhodia) [cationic density = 3.95 meq/g, MW ~243,000]
⁽¹¹⁾Polyquaternium 6 (Mirapol 100, available from Rhodia)
⁽¹²⁾Triquat (Rhodia, charge density = 6.07 meq/g)
⁽¹³⁾NHance 3269 (available from Aqualon/Hercules)
⁽¹⁴⁾Polymer LR400 (available from Amerchol/Dow)
⁽¹⁵⁾Cassia galactomannan (MW ~200,000, charge density = 0.7 or 3.0 meq/g)
⁽¹⁶⁾Thixcin R (Rheox)
⁽¹⁷⁾PEG 14M (Dow Chemical)
⁽¹⁸⁾ZPT (having an average particle size of about 2.5 micron, available from Arch)
⁽¹⁹⁾available from Bruggemann Chemical
⁽²⁰⁾Materials made by reported methods in Lagaly, G: et al. Inorg. Chem. 3, 32, 1209-1215 & Morioka, H; et al. Inorg chem.. 1999, 38, 4211-4216.
⁽²¹⁾Viscasil 330M (General Electric Silicones)
⁽²²⁾Dow Corning ® 1664 Emulsion (Dow Corning)
⁽²³⁾Dow Corning ® 2-1865 Microemulsion (Dow Corning)
⁽²⁴⁾Puresyn 6, MCP-1812 (Mobil)
⁽²⁵⁾Mobil P43 (Mobil)
⁽²⁶⁾Sipernat 22LS (Degussa)
⁽²⁷⁾MSS-500H (GE Silicones)
⁽²⁸⁾MSS-500N (GE Silicones)
⁽²⁹⁾Syloid 244FP Silica (Grace Davison)
⁽³⁰⁾Tospearl 240 (GE Silicones)
⁽³¹⁾Tospearl 3120 (GE Silicones)
⁽³²⁾Miranol Ultra L32 (Rhodia)
⁽³³⁾Brij 30
⁽³⁴⁾Varisoft 110 (Witco)

### 10. Other Ingredients

The present invention may, in some embodiments, further comprise additional optional components known or otherwise effective for use in hair care or personal care products. The concentration of such optional ingredients generally ranges from zero to about 25%, more typically from about 0.05% to about 20%, even more typically from about 0.1% to about 15%, by weight of the composition. Such optional components should also be physically and chemically compatible with the essential components described herein, and should not otherwise unduly impair product stability, aesthetics or performance.

Non-limiting examples of optional components for use in the present invention include anti-static agents, foam boosters, anti-dandruff agents in addition to the anti-dandruff agents described above, viscosity adjusting agents and thickeners, suspension materials (e.g. EGDS, thixins), pH adjusting agents (e.g. sodium citrate, citric acid, succinic acid, sodium succinate, sodium maleate, sodium glycolate, malic acid, glycolic acid, hydrochloric acid, sulfuric acid, sodium bicarbonate, sodium hydroxide, and sodium carbonate), preservatives (e.g. DMDM hydantoin), anti-microbial agents (e.g. triclosan or triclocarbon), dyes, organic solvents or diluents, pearlescent aids, perfumes, fatty alcohols, proteins, skin active agents, sunscreens, vitamins (such as retinoids including retinyl propionate, vitamin E such as tocopherol acetate, panthenol, and vitamin B3 compounds including niacinamide), emulsifiers, volatile carriers, select stability actives, styling polymers, organic styling polymers, silicone-grafted styling polymers, cationic spreading agents, pediculocides, foam boosters, viscosity modifiers and thickeners, polyalkylene glycols and combinations thereof.

Optional anti-static agents such as water-insoluble cationic surfactants may be used, typically in concentrations ranging from about 0.1% to about 5%, by weight of the composition. Such anti-static agents should not unduly interfere with the in-use performance and end-benefits of the anti-microbial composition; particularly, the anti-static agent should not interfere with the anionic surfactant. A specific non-limiting example of a suitable anti-static agents is tricetyl methyl ammonium chloride.

Optional foam boosters for use in the present invention described herein include fatty ester (e.g. C₈-C₂₂) mono- and di(C₁-C₅, especially C₁-C₃)alkanol amides. Specific non-limiting examples of such foam boosters include coconut monoethanolamide, coconut diethanolamide, and mixtures thereof.

Optional viscosity modifiers and thickeners may be used, typically in amounts effective for the anti-microbial compositions of the present invention to generally have an overall viscosity from about 1,000 csk to about 20,000 csk, preferably from about 3,000 csk to about 10,000 csk. Specific non-limiting examples of such viscosity modifiers and thickeners include: sodium chloride, sodium sulfate, and mixtures thereof.

### P. OTHER PREFERRED EMBODIMENTS

Other preferred embodiments of the present invention include the following:

An embodiment of the present invention, relates to the composition may be employed to treat a variety of conditions, including: athlete's foot, microbial infections, improving the appearance of a scalp, treating fungal infections, treating dandruff, treating diaper dermatitis and candidiasis, treating tinea capitis, treating yeast infections, treating onychomycosis. Preferably, such conditions are treated by applying a composition of the present invention to the affected area.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a) an effective amount of a particulate zinc material;
b) an effective amount of a surfactant including a surfactant with an anionic functional group;
c) an effective amount of a pyrithione or a polyvalent metal salt of a pyrithione;
d) from about 0.025% to about 5% by weight of a water soluble or dispersible, cationic, non-crosslinked, conditioning homopolymer having a cationic charge density of from about 2 meq/gm to about 10 meq/gm; and
e) from about 20% to about 95% of an aqueous carrier, by weight of said composition.

2. A composition according to claim 2 wherein the pyrithione or polyvalent metal salt of pyrithione is zinc pyrithione.

3. The composition of claim 1 wherein the cationic charge density of the cationic polymer is from about 3 meq/gm to about 10 meq/gm.

4. The composition of claim 1 wherein the cationic charge density of the cationic polymer is from about 4 meq/gm to about 7 meq/gm.

5. The composition of claim 1 wherein the cationic polymer has an average molecular weight of from about 1,000 to about 5,000,000.

6. The composition of claim 1 wherein said cationic polymer promotes the formation of a microscopic-phase separation of lyotropic liquid crystals in said composition; the liquid crystals exhibiting birefringence.

7. A composition according to claim 1 comprising lytropic liquid crystals that aids in the deposition of particles.

8. A composition according to claim 1, wherein said synthetic cationic polymer comprises monomers selected from the group consisting of dimethylaminoethyl (meth)acrylate, dimethylaminopropyl(meth)acrylate, ditertiobutylaminoethyl (meth)acrylate, dimethylaminomethyl(meth)acrylamide, dimethylaminopropyl (meth)acrylamide; ethylenimine, vinylamine, 2-vinylpyridine, 4-vinylpyridine, trimethylammonium ethyl(meth)acrylate chloride, trimethylammonium ethyl (meth)acrylate methyl sulphate, dimethylammonium ethyl(meth)acrylate benzyl chloride, 4-benzoylbenzyl dimethylammonium ethyl acrylate chloride, trimethyl ammonium ethyl(meth)acrylamido chloride, trimethyl ammonium propyl (meth)acrylamido chloride, vinylbenzyl trimethyl ammonium chloride, diallyldimethyl ammonium chloride, trimethylammonium ethyl(meth)acrylate chloride, trimethylammonium ethyl(meth)acrylate methyl sulphate, dimethylammonium ethyl(meth)acrylate benzyl chloride, 4-benzoylbenzyl dimethylammonium ethyl acrylate chloride, trimethyl ammonium ethyl (meth)acrylamido chloride, trimethyl ammonium propyl(meth)acrylamido chloride, vinylbenzyl trimethyl ammonium chloride and trimethyl ammonium propyl(meth)acrylamido chloride.

9. A composition according to claim 1 wherein the particulate zinc material has a relative zinc lability of greater than about 15%.

10. A composition according to claim 1 wherein the composition comprises less than 5.5 micromoles of a zinc binding material per gram of the particulate zinc material/per m²/gram surface area of the particulate zinc material.

11. A composition according to claim 10 wherein the zinc binding material is selected from the group comprising laurate, citrate, valerate, oxalate, tartrate, iodate, thiocyanate, cyanide, sulfide, pyrophosphate, phosphate and mixtures thereof.

12. A composition according to claim 11 wherein the zinc binding material is laurate.

13. A composition according to claim 1 wherein the detersive surfactant with an anionic functional group is about 1% to about 50% of the total composition.

14. A composition according to claim 2 wherein the ZPT is present from about 0.01% to about 5%.

15. A composition according to claim 1 wherein the detersive surfactant is present from about 2% to about 50%.

16. A composition according to claim 15 wherein the detersive surfactant is selected from the group consisting of anionic, cationic, nonionic, amphoteric or zwitterionic.

17. A composition according to claim 18 wherein the surfactant is anionic.

18. A composition according to claim 1 wherein the pH is greater than about 6.5.

19. A composition according to claim 18 wherein the pH is from about 6.8 to about 9.5

20. A composition according to claim 19 wherein the pH is from about 6.8 to about 8.5.

21. A composition according to claim 1 wherein the particulate zinc material has a relative zinc lability of greater than about 20%.

22. A composition according to claim 1 wherein the particulate zinc material has a relative zinc lability of greater than about 25%.

23. A composition according to claim 1 wherein the particulate zinc material is selected from the group consisting of inorganic materials, natural zinc sources, ores, minerals, organic salts, polymeric salts, or physically adsorbed from material and mixtures thereof.

24. A composition according to claim 23 wherein the inorganic materials is selected from the group consisting of zinc aluminate, zinc carbonate, zinc oxide, calamine, zinc phosphate, zinc selenide, zinc sulfide, zinc silicates, zinc silicofluoride, zinc borate, or zinc hydroxide and zinc hydroxy sulfate, zinc-containing layered material and mixtures thereof.

25. A composition according to claim 24 the zinc-containing layered material is selected from the group consisting of basic zinc carbonate, zinc carbonate hydroxide, hydrozincite, zinc copper carbonate hydroxide, aurichalcite, copper zinc carbonate hydroxide, rosasite, phyllosilicate containing zinc ions, layered double hydroxide, hydroxy double salts and mixtures thereof.

26. A composition according to claim 25 wherein the zinc-containing layered material is selected from the group consisting of zinc carbonate hydroxide, hydrozincite, basic zinc carbonate and mixtures thereof.

27. A composition according to claim 26 wherein the zinc-containing layered material is hydrozincite or basic zinc carbonate.

28. A composition according to claim 27 wherein the zinc-containing layered material is basic zinc carbonate.

29. A composition according to claim 1 wherein the composition further comprises an additional cationic polymer.

30. A composition according to claim 29 wherein the additional cationic polymer is a naturally derived cationic polymer.

31. A composition according to claim 29 wherein the additional cationic polymer is a synthetic polymer having a charge density of less than about 2.0 meq/gm.

32. A composition according to claim 30 wherein said naturally derived cationic polymer is selected from the group consisting of celluloses, starches, guars, and non-guar galactomannans.

33. A composition according to claim 32 wherein the cationic polymer has a trimethylamine level of less than about 45 ppm.

34. A composition according to claim 33 wherein the cationic polymer is a cationic guar.

35. A composition according to claim 1 wherein the composition further comprises a conditioning agent.

36. A composition according to claim 35 wherein the conditioning agent is a silicone.

37. A composition according to claim 1 wherein the composition further comprises a suspending agent.

38. A composition according to claim 37 wherein the suspending agent is selected from the group consisting of crystalline suspending agent, polymeric suspending agent or mixtures thereof.

39. A composition according to claim 38 wherein the suspending agent is a crystalline suspending agent.

40. A method of treating dandruff comprising the use of the composition of claim 1.

41. A composition comprising:
a) an effective amount of a particulate zinc material;
b) an effective amount of a surfactant including a surfactant with an anionic functional group;
c) an effective amount of a pyrithione or a polyvalent metal salt of a pyrithione;
d) from about 0.025% to about 5% by weight of a water soluble or dispersible, cationic, non-crosslinked, conditioning copolymer having a cationic charge density of from about 2 meq/gm to about 10 meq/gm; and
e) from about 20% to about 95% of an aqueous carrier, by weight of said composition.

42. A composition according to claim 41 wherein the water soluble or dispersible, cationic, non-crosslinked, conditioning copolymer having a cationic charge density of from about 2 meq/gm to about 7 meq/gm.

43. A composition comprising:
a) an effective amount of a particulate zinc material;
b) an effective amount of a surfactant including a surfactant with an anionic functional group;
c) an effective amount of a pyrithione or a polyvalent metal salt of a pyrithione;
d) from about 0.025% to about 5% by weight of a water soluble or dispersible, cationic, non-crosslinked, conditioning homopolymer having an average molecular weight of from about 500,000 to about 5,000,000; and
e) from about 20% to about 95% of an aqueous carrier, by weight of said composition.

44. A composition comprising:
a) an effective amount of a particulate zinc material;
b) an effective amount of a surfactant including a surfactant with an anionic functional group;
c) an effective amount of a pyrithione or a polyvalent metal salt of a pyrithione;
d. from about 0.025% to about 5% by weight of a water soluble or dispersible, cationic, non-crosslinked, conditioning copolymer comprising:
i. one or more cationic monomer units, and
ii. one or more nonionic or monomer units bearing a terminal negative charge
wherein said copolymer has a positive charge, a cationic charge density of from about 2 meq/gm to about 10 meq/gm, and an average molecular weight of from about 1,000 to about 5,000,000; and
e. from about 20% to about 95% of an aqueous carrier, by weight of said composition.
